(19) United States
(12) Patent Application Publication
(10) Pub. No.: US 2002/0164817 A1
(43) Pub. Date: Nov. 7, 2002

(54) Stimulable phosphor sheet and method for reading biochemical analysis data recorded in stimulable phosphor sheet

(75) Inventor: Keiko Neriishi, Kanagawa (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/117,223

(22) Filed: Apr. 8, 2002

(30) Foreign Application Priority Data
Apr. 9, 2001 (JP) 2001-110261

(51) Int. Cl. C12Q 1/68; G03C 1/00; G01N 21/76

(52) U.S. Cl. 436/172; 435/6; 430/496; 436/164; 436/57; 435/7.1; 435/288.4; 435/288.7; 422/82.05; 422/82.08; 422/99; 422/102

(57) ABSTRACT

A stimulable phosphor sheet includes a support formed with a plurality of stimulable phosphor layer regions spaced apart from each other and a plurality of additional stimulable phosphor layer regions spaced apart from the plurality of stimulable phosphor layer regions. According to the thus constituted stimulable phosphor sheet, it is possible to produce biochemical analysis data having excellent quantitative characteristics with high resolution even in the case of forming at a high density on the surface of a carrier a plurality of spot-like regions containing specific binding substances which can specifically bind with a substance derived from a living organism and whose sequence, base length, composition and the like are known, and specifically binding a substance derived from a living organism labeled with a radioactive labeling substance with specific binding substances contained in the plurality of spot-like regions, thereby selectively labeling the plurality of spot-like regions.

### BACKGROUND OF THE INVENTION

The present invention relates to a stimulable phosphor sheet and a method for reading biochemical analysis data recorded in a stimulable phosphor sheet and, particularly, to a stimulable phosphor sheet and a method for reading biochemical analysis data recorded in a stimulable phosphor sheet which can produce biochemical analysis data having excellent quantitative characteristics with high resolution even in the case of forming at a high density on the surface of a carrier a plurality of spot-like regions containing specific binding substances which can specifically bind with a substance derived from a living organism and whose sequence, base length, composition and the like are known, and specifically binding a substance derived from a living organism labeled with a radioactive labeling substance with the specific binding substances contained in the plurality of spot-like regions, thereby selectively labeling the plurality of spot-like regions.

### DESCRIPTION OF THE PRIOR ART

An autoradiographic analyzing system using as a detecting material for detecting radiation a stimulable phosphor which can absorb, store and record the energy of radiation when it is irradiated with radiation and which, when it is then stimulated by an electromagnetic wave having a specified wavelength, can release stimulated emission whose light amount corresponds to the amount of radiation with which it was irradiated is known, which comprises the steps of introducing a radioactively labeled substance into an organism, using the organism or a part of the tissue of the organism as a specimen, superposing the specimen and a stimulable phosphor sheet formed with a stimulable phosphor layer for a certain period of time, storing and recording radiation energy in a stimulable phosphor contained in the stimulable phosphor layer, scanning the stimulable phosphor layer with an electromagnetic wave to excite the stimulable phosphor, photoelectrically detecting the stimulated emission released from the stimulable phosphor to produce digital image signals, effecting image processing on the obtained digital image signals, and reproducing an image on displaying means such as a CRT or the like or a photographic film (see, for example, Japanese Patent Publication No. 1-60784, Japanese Patent Publication No. 1-60782, Japanese Patent Publication No. 4-3952 and the like).

Unlike the system using a photographic film, according to the autoradiographic analyzing system using the stimulable phosphor as a detecting material, development, which is chemical processing, becomes unnecessary. Further, it is possible reproduce a desired image by effecting image processing on the obtained image data and effect quantitative analysis using a computer. Use of a stimulable phosphor in these processes is therefore advantageous.

On the other hand, a fluorescence analyzing system using a fluorescent substance as a labeling substance instead of a radioactive labeling substance in the autoradiographic analyzing system is known. According to this system, it is possible to study a genetic sequence, study the expression level of a gene, and to effect separation or identification of protein or estimation of the molecular weight or properties of protein or the like. For example, this system can perform a process including the steps of distributing a plurality of DNA fragments on a gel support by means of electrophoresis after a fluorescent dye was added to a solution containing a plurality of DNA fragments to be distributed, or distributing a plurality of DNA fragments on a gel support containing a fluorescent dye, or dipping a gel support on which a plurality of DNA fragments have been distributed by means of electrophoresis in a solution containing a fluorescent dye, thereby labeling the electrophoresed DNA fragments, exciting the fluorescent dye by a stimulating ray to cause it to release fluorescent light, detecting the released fluorescent light to produce an image and detecting the distribution of the DNA fragments on the gel support. This system can also perform a process including the steps of distributing a plurality of DNA fragments on a gel support by means of electrophoresis, denaturing the DNA fragments, transferring at least a part of the denatured DNA fragments onto a transfer support such as a nitrocellulose support by the Southern-blotting method, hybridizing a probe prepared by labeling target DNA and DNA or RNA complementary thereto with the denatured DNA fragments, thereby selectively labeling only the DNA fragments complementary to the probe DNA or probe RNA, exciting the fluorescent dye by a stimulating ray to cause it to release fluorescent light, detecting the released fluorescent light to produce an image and detecting the distribution of the target DNA on the transfer support. This system can further perform a process including the steps of preparing a DNA probe complementary to DNA containing a target gene labeled by a labeling substance, hybridizing it with DNA on a transfer support, combining an enzyme with the complementary DNA labeled by a labeling substance, causing the enzyme to contact a fluorescent substance, transforming the fluorescent substance to a fluorescent substance having fluorescent light releasing property, exciting the thus produced fluorescent substance by a stimulating ray to release fluorescent light, detecting the fluorescent light to produce an image and detecting the distribution of the target DNA on the transfer support. This fluorescence detecting system is advantageous in that a genetic sequence or the like can be easily detected without using a radioactive substance.

Similarly, there is known a chemiluminescence detecting system comprising the steps of fixing a substance derived from a living organism such as a protein or a nucleic acid sequence on a support, selectively labeling the substance derived from a living organism with a labeling substance which generates chemiluminescent emission when it contacts a chemiluminescent substrate, contacting the substance derived from a living organism and selectively labeled with the labeling substance and the chemiluminescent substrate, photoelectrically detecting the chemiluminescent emission in the wavelength of visible light generated by the contact of the chemiluminescent substrate and the labeling substance to produce digital image signals, effecting image processing thereon, and reproducing a chemiluminescent image on a display means such as a CRT or a recording material such as a photographic film, thereby obtaining information relating to the high molecular substance such as genetic information.

Further, a micro-array analyzing system has been recently developed, which comprises the steps of using a spotting device to drop at different positions on the surface of a carrier such as a slide glass plate, a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substances using a hybridization method or the like with a substance derived from a living organism such as a cell, virus, hormone, tumor marker, enzyme, antibody, antigen, abzyme, other protein, a nuclear acid, cDNA, DNA or mRNA by extraction, isolation or the like and optionally further subjected to chemical processing, chemical modification or the like and which is labeled with a labeling substance such as a fluorescent substance, dye or the like, thereby forming a micro-array, irradiating the micro-array with a stimulating ray, photoelectrically detecting light such as fluorescence emission released from a labeling substance such as a fluorescent substance, dye or the like, and analyzing the substance derived from a living organism. This micro-array analyzing system is advantageous in that a substance derived from a living organism can be analyzed in a short time period by forming a number of spots of specific binding substances at different positions of the surface of a carrier such as a slide glass plate at high density and hybridizing them with a substance derived from a living organism and labeled with a labeling substance.

In addition, a macro-array analyzing system using a radioactive labeling substance as a labeling substance has been further developed, which comprises the steps of using a spotting device to drop at different positions on the surface of a carrier such as a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substance using a hybridization method or the like with a substance derived from a living organism such as a cell, virus, hormone, tumor marker, enzyme, antibody, antigen, abzyme, other protein, a nuclear acid, cDNA, DNA or mRNA by extraction, isolation or the like and optionally further subjected to chemical processing, chemical modification or the like and which is labeled with a radioactive labeling substance, thereby forming a macro-array, superposing the macro-array and a stimulable phosphor sheet formed with a stimulable phosphor layer, exposing the stimulable phosphor layer to a radioactive labeling substance, irradiating the stimulable phosphor layer with a stimulating ray to excite the stimulable phosphor, photoelectrically detecting the stimulated emission released from the stimulable phosphor to produce biochemical analysis data, and analyzing the substance derived from a living organism.

However, in the macro-array analyzing system using a radioactive labeling substance as a labeling substance, when the stimulable phosphor layer is exposed to a radioactive labeling substance, since the radiation energy of the radioactive labeling substance contained in spot-like regions formed on the surface of a carrier such as a membrane filter is very large, electron beams (β rays) released from the radioactive labeling substance contained in the individual spot-like regions are scattered in the carrier such as a membrane filter, thereby impinging on regions of the stimulable phosphor layer that should be exposed only to the radioactive labeling substance contained in neighboring spot-like regions, or electron beams released from radioactive labeling substance adhering to the surface of the carrier such as a membrane filter between neighboring spot-like regions impinge on the stimulable phosphor layer, to generate noise in biochemical analysis data produced by photoelectrically detecting stimulated emission, thus making data of neighboring spot-like regions hard to separate and lowering resolution, and to lower the accuracy of biochemical analysis when a substance derived from a living organism is analyzed by quantifying the radiation amount of each spot. The degradation of the resolution and accuracy of biochemical analysis is particularly pronounced when spots are formed close to each other at high density.

### SUMMARY OF THE INVENTION

It is therefore an object of the present invention to provide a stimulable phosphor sheet and a method for reading biochemical analysis data recorded in a stimulable phosphor sheet which can produce biochemical analysis data having excellent quantitative characteristics with high resolution even in the case of forming at a high density on the surface of a carrier a plurality of spot-like regions containing specific binding substances which can specifically bind with a substance derived from a living organism and whose sequence, base length, composition and the like are known, and specifically binding a substance derived from a living organism labeled with a radioactive labeling substance with specific binding substances contained in the plurality of spot-like regions, thereby selectively labeling the plurality of spot-like regions.

The above other objects of the present invention can be accomplished by a stimulable phosphor sheet including a support formed with a plurality of stimulable phosphor layer regions spaced apart from each other and at least one additional stimulable phosphor layer region spaced apart from the plurality of stimulable phosphor layer regions.

According to the present invention, in the case of forming at a high density on the surface of a carrier such as a membrane filter a plurality of spot-like regions containing specific binding substances which can specifically bind with a substance derived from a living organism and whose sequence, base length, composition and the like are known, and specifically binding a substance derived from a living organism labeled with a radioactive labeling substance with the specific binding substances contained in the plurality of spot-like regions, thereby selectively labeling the plurality of spot-like regions, electron beams (β rays) released from the radioactive labeling substance contained in the individual spot-like regions when the stimulable phosphor sheet is superposed on the carrier to expose the plurality of stimulable phosphor layer regions of the stimulable phosphor sheet to the radioactive labeling substance selectively contained in the plurality of spot-like regions of the carrier can be effectively prevented from entering stimulable phosphor layer regions other than that to be exposed to electron beams (β rays) released from the radioactive labeling substance contained in the spot-like region and, therefore, it is possible to produce biochemical analysis data having an excellent quantitative characteristic with high resolution by scanning the plurality of the thus exposed stimulable phosphor layer regions with a stimulating ray and photoelectrically detecting stimulated emission released from the plurality of stimulable phosphor layer regions.

Further, when the carrier constituted as a membrane filter formed with a plurality of spot-like regions selectively containing a radioactive labeling substance and the stimulable phosphor sheet formed with the plurality of stimulable phosphor regions are superposed, thereby exposing the plurality of stimulable phosphor regions to the radioactive labeling substance selectively contained in the plurality of spot-like regions, since not only electron beams (β rays) released from the radioactive labeling substance contained in the plurality of spot-like regions but also electron beams (β rays) released from radioactive labeling substance adhering to the surface of the carrier during hybridization and remaining even after washing, ambient radiation and the like enter the plurality of stimulable phosphor regions formed in the support of the stimulable phosphor sheet, background noise caused by radioactive labeling substance adhering to the surface of the carrier during hybridization and remaining even after washing, ambient radiation and the like entering the plurality of stimulable phosphor regions formed in the support of the stimulable phosphor sheet is inevitably generated in biochemical analysis data obtained by scanning the plurality of exposed stimulable phosphor regions of the stimulable phosphor sheet with a stimulating ray and photoelectrically detecting stimulated emission released from the plurality of stimulable phosphor regions. However, according to the present invention, since the support of the stimulable phosphor sheet is further formed with at least one additional stimulable phosphor region spaced apart from the plurality of stimulable phosphor regions and electron beams (β rays) released from the radioactive labeling substance contained in the plurality of spot-like regions do not enter the at least one additional stimulable phosphor region so that the at least one additional stimulable phosphor region is exposed only to electron beams (β rays) released from radioactive labeling substance adhering to the surface of the carrier during hybridization and remaining even after washing, ambient radiation and the like, background noise data can be obtained by scanning the at least one additional stimulable phosphor region with a stimulating ray and photoelectrically detecting stimulated emission released therefrom. Therefore, it is possible to produce biochemical analysis data free of background noise by subtracting the data obtained by scanning the at least one additional stimulable phosphor region with a stimulating ray and photoelectrically detecting stimulated emission released therefrom from biochemical analysis data obtained by scanning the plurality of exposed stimulable phosphor regions of the stimulable phosphor sheet with a stimulating ray and photoelectrically detecting stimulated emission released therefrom.

The above and other objects of the present invention can be also accomplished by a method for reading biochemical analysis data recorded in a stimulable phosphor sheet comprising the steps of superposing a stimulable phosphor sheet including a support formed with a plurality of stimulable phosphor layer regions spaced apart from each other and at least one additional stimulable phosphor layer region spaced apart from the plurality of stimulable phosphor layer regions and a biochemical analysis unit including a plurality of spot-like regions formed by spotting specific binding substances whose sequence, base length, composition and the like are known and specifically binding a substance derived from a living organism labeled with a radioactive labeling substance with the specific binding substances, thereby selectively labeling the the plurality of spot-like regions with the radioactive labeling substance, exposing the plurality of stimulable phosphor layer regions of the stimulable phosphor sheet to the radioactive labeling substance selectively contained in the plurality of spot-like regions, irradiating the plurality of stimulable phosphor layer regions and the at least one additional stimulable phosphor layer region of the stimulable phosphor sheet with a stimulating ray, thereby exciting stimulable phosphor contained in the plurality of stimulable phosphor layer regions and the at least one additional stimulable phosphor layer region, photoelectrically detecting stimulated emission released from the stimulable phosphor to produce analog data, digitizing the analog data to produce digital data and subtracting digital data obtained by irradiating the at least one additional stimulable phosphor layer region with the stimulating ray and photoelectrically detecting stimulated emission released therefrom from digital data obtained by irradiating the plurality of stimulable phosphor layer regions with the stimulating ray and photoelectrically detecting stimulated emission released therefrom, thereby producing biochemical analysis data.

When the stimulable sheet including the support formed with a plurality of stimulable phosphor layer regions spaced apart from each other and the biochemical analysis unit including the plurality of spot-like regions formed by spotting specific binding substances whose sequence, base length, composition and the like are known and specifically binding a substance derived from a living organism labeled with a radioactive labeling substance with the specific binding substances, thereby selectively labeling the plurality of spot-like regions with the radioactive labeling substance, and the plurality of stimulable phosphor layer regions formed in the stimulable phosphor sheet are exposed to the radioactive labeling substance selectively contained in the plurality of spot-like regions formed in the biochemical analysis unit, not only electron beams (β rays) released from the radioactive labeling substance contained in the plurality of spot-like regions but also electron beams (β rays) released from radioactive labeling substance adhering to regions other than the plurality spot-like regions on the surface of the carrier during hybridization and remaining even after washing, ambient radiation and the like enter the plurality of stimulable phosphor regions formed in the support of the stimulable phosphor sheet. As a result, background noise caused by radioactive labeling substance adhering to the regions other than the plurality of spot-like regions on the surface of the carrier during hybridization and remaining even after washing, ambient radiation and the like entering the plurality of stimulable phosphor regions formed in the support of the stimulable phosphor sheet is inevitably generated in biochemical analysis data obtained by scanning the plurality of exposed stimulable phosphor regions of the stimulable phosphor sheet with a stimulating ray and photoelectrically detecting stimulated emission released therefrom. However, according to the present invention, since the support of the stimulable phosphor sheet is further formed with at least one additional stimulable phosphor region spaced apart from the plurality of stimulable phosphor regions and electron beams (β rays) released from the radioactive labeling substance contained in the plurality of spot-like regions do not enter the at least one additional stimulable phosphor region so that the at least one additional stimulable phosphor region is exposed only to electron beams (β rays) released from radioactive labeling substance adhering to the regions other than the plurality of spot-like regions on the surface of the carrier during hybridization and remaining even after washing, ambient radiation and the like, background noise data can be obtained by scanning the at least one additional stimulable phosphor region with a stimulating ray and photoelectrically detecting stimulated emission released therefrom. Therefore, it is possible to produce biochemical analysis data free of background noise by subtracting data obtained by superposing the stimulable phosphor sheet including a support formed with the plurality of stimulable phosphor layer regions spaced apart from each other and the at least one additional stimulable phosphor layer region spaced apart from the plurality of stimulable phosphor layer regions and the biochemical analysis unit including the plurality of spot-like regions formed by spotting specific binding substances whose sequence, base length, composition and the like are known and specifically binding a substance derived from a living organism labeled with the radioactive labeling substance with the specific binding substances, thereby selectively the plurality of spot-like regions with the radioactive labeling substance, exposing the plurality of stimulable phosphor layer regions formed in the stimulable phosphor sheet to the radioactive labeling substance selectively contained in the plurality of spot-like regions formed in the biochemical analysis unit, irradiating the plurality of stimulable phosphor layer regions and the at least one additional stimulable phosphor layer region of the stimulable phosphor sheet with a stimulating ray, thereby exciting stimulable phosphor contained in the plurality of stimulable phosphor layer regions and the at least one additional stimulable phosphor layer region, photoelectrically detecting stimulated emission released from the stimulable phosphor to produce analog data, digitizing the analog data to produce digital data and subtracting digital data obtained by irradiating the at least one additional stimulable phosphor layer region with the stimulating ray and photoelectrically detecting stimulated emission released therefrom from digital data obtained by irradiating the plurality of stimulable phosphor layer regions with the stimulating ray and photoelectrically detecting stimulated emission released therefrom.

In a preferred aspect of the present invention, the support of the stimulable phosphor sheet is formed with a plurality of holes spaced apart from each other and the plurality of stimulable phosphor layer regions are formed by charging stimulable phosphor in the plurality of holes.

In a further preferred aspect of the present invention, the support of the stimulable phosphor sheet is formed with a plurality of through-holes spaced apart from each other and the plurality of stimulable phosphor layer regions are formed by charging stimulable phosphor in the plurality of through-holes.

In another preferred aspect of the present invention, the support of the stimulable phosphor sheet is formed with a plurality of through-holes spaced apart from each other and the plurality of stimulable phosphor layer regions of the stimulable phosphor sheet are formed by pressing a stimulable phosphor membrane containing stimulable phosphor in the through-holes.

In another preferred aspect of the present invention, the support of the stimulable phosphor sheet is formed with a plurality of recesses spaced apart from each other and the plurality of stimulable phosphor layer regions are formed by charging stimulable phosphor in the plurality of recesses.

In a preferred aspect of the present invention, the plurality of stimulable phosphor layer regions of the stimulable phosphor sheet are formed on the surface of the support of the stimulable phosphor sheet.

In a preferred aspect of the present invention, the plurality of stimulable phosphor layer regions of the stimulable phosphor sheet are dot-like formed in the support.

In a preferred aspect of the present invention, each of the plurality of stimulable phosphor layer regions of the stimulable phosphor sheet is formed substantially circular.

In a preferred aspect of the present invention, a plurality of the additional stimulable phosphor layer regions are dot-like formed in the support of the stimulable phosphor sheet.

In a further preferred aspect of the present invention, a plurality of the additional stimulable phosphor layer regions are dot-like formed in the support of the stimulable phosphor sheet between at least some of the plurality of stimulable phosphor layer regions.

Although background noise differs between different positions on the surface of the stimulable phosphor sheet, namely, the individual stimulable phosphor layer regions, according to this preferred aspect of the present invention, since the plurality of the additional stimulable phosphor layer regions are dot-like formed in the support between at least some of the plurality of stimulable phosphor layer regions, even if background noise varies between different positions on the surface of the stimulable phosphor sheet, it is possible to produce biochemical analysis data free of background noise with high accuracy.

In another preferred aspect of the present invention, the at least one additional stimulable phosphor layer region of the stimulable phosphor sheet is formed in a stripe shape in the support.

In a further preferred aspect of the present invention, the at least one additional stimulable phosphor layer regions of the stimulable phosphor sheet is formed in a stripe shape in the support between at least some of the plurality of stimulable phosphor layer regions.

Although background noise differs between different positions on the surface of the stimulable phosphor sheet, namely, the individual stimulable phosphor layer regions, according to this preferred aspect of the present invention, since the at least one additional stimulable phosphor layer regions of the stimulable phosphor sheet is formed in a stripe shape in the support between at least some of the plurality of stimulable phosphor layer regions, even if background noise varies between different positions on the surface of the stimulable phosphor sheet, it is possible to produce biochemical analysis data free of background noise with high accuracy.

In a preferred aspect of the present invention, each of the additional stimulable phosphor layer regions of the stimulable phosphor sheet is formed substantially circular.

In a preferred aspect of the present invention, each of the additional stimulable phosphor layer regions of the stimulable phosphor sheet is formed so as to have a smaller size than that of each of the plurality of stimulable phosphor layer regions.

In a preferred aspect of the present invention, the support of the stimulable phosphor sheet is formed of a material capable of attenuating radiation energy.

According to this preferred aspect of the present invention, in the case of forming at a high density on the surface of a carrier such as a membrane filter a plurality of spot-like regions containing specific binding substances which can specifically bind with a substance derived from a living organism and whose sequence, base length, composition and the like are known, and specifically binding a substance derived from a living organism labeled with a radioactive labeling substance with specific binding substances contained in the plurality of spot-like regions, thereby selectively labeling the plurality of spot-like regions, when a plurality of stimulable phosphor regions are to be exposed to a radiographic labeling substance selectively contained in the plurality of spot-like regions by superposing the stimulable phosphor sheet on the carrier, it is possible to effectively prevent electron beams (β rays) released from the radioactive labeling substance contained in the individual spot-like regions from impinging on stimulable phosphor regions other than the stimulable phosphor regions to be exposed to electron beams (β rays) released from the radioactive labeling substance contained in the spot-like region by forming the plurality of stimulable phosphor regions in the support in the same pattern as that of the plurality of spot-like regions formed on the carrier and, therefore, to produce biochemical analysis data having excellent quantitative characteristics with high resolution by scanning the plurality of exposed stimulable phosphor regions with a stimulating ray and photoelectrically detecting stimulated emission released from the plurality of stimulable phosphor regions.

In a preferred aspect of the present invention, the support of the stimulable phosphor sheet is made of a material of reducing the energy of radiation to ⅕ or less when the radiation travels in the support by a distance equal to that between neighboring stimulable phosphor layer regions.

In a further preferred aspect of the present invention, the support of the stimulable phosphor sheet is made of a material of reducing the energy of radiation to 1/10 or less when the radiation travels in the support by a distance equal to that between neighboring stimulable phosphor layer regions.

In a further preferred aspect of the present invention, the support of the stimulable phosphor sheet is made of a material of reducing the energy of radiation to 1/50 or less when the radiation travels in the support by a distance equal to that between neighboring stimulable phosphor layer regions.

In a further preferred aspect of the present invention, the support of the stimulable phosphor sheet is made of a material of reducing the energy of radiation to 1/100 or less when the radiation travels in the support by a distance equal to that between neighboring stimulable phosphor layer regions.

In a further preferred aspect of the present invention, the support of the stimulable phosphor sheet is made of a material of reducing the energy of radiation to 1/500 or less when the radiation travels in the support by a distance equal to that between neighboring stimulable phosphor layer regions.

In a further preferred aspect of the present invention, the support of the stimulable phosphor sheet is made of a material of reducing the energy of radiation to 1/1,000 or less when the radiation travels in the support by a distance equal to that between neighboring stimulable phosphor layer regions.

In the present invention, material for forming the support of the stimulable phosphor has preferably a property capable of attenuating radiation energy but is not particularly limited. The material for forming the support of the stimulable phosphor may be of any type of inorganic compound material or organic compound material and the support of the stimulable phosphor sheet preferably formed of metal material, ceramic material or plastic material.

Illustrative examples of inorganic compound materials preferably usable for forming the support of the stimulable phosphor sheet and capable of attenuating radiation energy in the present invention include metals such as gold, silver, copper, zinc, aluminum, titanium, tantalum, chromium, iron, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. These may have either a monocrystal structure or a polycrystal sintered structure such as amorphous, ceramic or the like.

In the present invention, a high molecular compound is preferably used as an organic compound material preferably usable for forming the support of the stimulable phosphor sheet and capable of attenuating radiation energy. Illustrative examples of high molecular compounds preferably usable for forming the support of the stimulable phosphor sheet in the present invention include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifuluoroethylene; polycarbonate; polyesters such as polyethylene naphthalate, polyethylene terephthalate and the like; nylons such as nylon-6, nylon-6,6, nylon-4, 10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Since the capability of attenuating radiation energy generally increases as specific gravity increases, the support of the stimulable phosphor sheet is preferably formed of a compound material or a composite material having specific gravity of 1.0 g/cm³ or more and more preferably formed of a compound material or a composite material having specific gravity of 1.5 g/cm³ to 23 g/cm³.

In a preferred aspect of the present invention, the support of the stimulable phosphor sheet is formed with 10 or more stimulable phosphor layer regions.

In a further preferred aspect of the present invention, the support of the stimulable phosphor sheet is formed with 50 or more stimulable phosphor layer regions.

In a further preferred aspect of the present invention, the support of the stimulable phosphor sheet is formed with 100 or more stimulable phosphor layer regions.

In a further preferred aspect of the present invention, the support of the stimulable phosphor sheet is formed with 500 or more stimulable phosphor layer regions.

In a further preferred aspect of the present invention, the support of the stimulable phosphor sheet is formed with 1,000 or more stimulable phosphor layer regions.

In a further preferred aspect of the present invention, the support of the stimulable phosphor sheet is formed with 5,000 or more stimulable phosphor layer regions.

In a further preferred aspect of the present invention, the support of the stimulable phosphor sheet is formed with 10,000 or more stimulable phosphor layer regions.

In a further preferred aspect of the present invention, the support of the stimulable phosphor sheet is formed with 50,000 or more stimulable phosphor layer regions.

In a further preferred aspect of the present invention, the support of the stimulable phosphor sheet is formed with 10,0000 or more stimulable phosphor layer regions.

In a preferred aspect of the present invention, each of the plurality of stimulable phosphor layer regions is formed in the support of the stimulable phosphor sheet to have a size of less than 5 mm².

In a further preferred aspect of the present invention, each of the plurality of stimulable phosphor layer regions is formed in the support of the stimulable phosphor sheet to have a size of less than 1 mm².

In a further preferred aspect of the present invention, each of the plurality of stimulable phosphor layer regions is formed in the support of the stimulable phosphor sheet to have a size of less than 0.5 mm².

In a further preferred aspect of the present invention, each of the plurality of stimulable phosphor layer regions is formed in the support of the stimulable phosphor sheet to have a size of less than 0.1 mm².

In a further preferred aspect of the present invention, each of the plurality of stimulable phosphor layer regions is formed in the support of the stimulable phosphor sheet to have a size of less than 0.05 mm².

In a further preferred aspect of the present invention, each of the plurality of stimulable phosphor layer regions is formed in the support of the stimulable phosphor sheet to have a size of less than 0.01 mm².

In the present invention, the density of the stimulable phosphor layer regions formed in the stimulable phosphor sheet can be determined depending upon the material of the support, the kind of electron beam released from the radioactive labeling substance and the like.

In a preferred aspect of the present invention, the plurality of stimulable phosphor layer regions are formed in the stimulable phosphor sheet at a density of 10 or more per cm².

In a further preferred aspect of the present invention, the plurality of stimulable phosphor layer regions are formed in the stimulable phosphor sheet at a density of 50 or more per cm².

In a further preferred aspect of the present invention, the plurality of stimulable phosphor layer regions are formed in the stimulable phosphor sheet at a density of 100 or more per cm².

In a further preferred aspect of the present invention, the plurality of stimulable phosphor layer regions are formed in the stimulable phosphor sheet at a density of 500 or more per cm².

In a further preferred aspect of the present invention, the plurality of stimulable phosphor layer regions are formed in the stimulable phosphor sheet at a density of 1,000 or more per cm².

In a further preferred aspect of the present invention, the plurality of stimulable phosphor layer regions are formed in the stimulable phosphor sheet at a density of 5,000 or more per cm².

In a further preferred aspect of the present invention, the plurality of stimulable phosphor layer regions are formed in the stimulable phosphor sheet at a density of 10,000 or more per cm².

In a preferred aspect of the present invention, the plurality of stimulable phosphor layer regions are formed according in a regular pattern in the stimulable phosphor sheet.

In a preferred aspect of the present invention, the plurality of stimulable phosphor layer regions of the stimulable phosphor sheet are formed in the support in a regular pattern.

According to this preferred aspect of the present invention, since the plurality of stimulable phosphor layer regions of the stimulable phosphor sheet are formed in the support in a regular pattern, it is possible to expose each stimulable phosphor layer region to the radioactive labeling substance contained in the corresponding spot-like region by forming the spot-like regions containing specific binding substances on the surface of the carrier such as a membrane filter in the same pattern as that of the plurality of stimulable phosphor layer regions and to produce biochemical analysis data having an excellent quantitative characteristic with high resolution.

The stimulable phosphor usable in the present invention may be of any type insofar as it can store radiation energy or electron beam energy and can be stimulated by an electromagnetic wave to release the radiation energy or the electron beam energy stored therein in the form of light. More specifically, preferably employed stimulable phosphors include alkaline earth metal fluorohalide phosphors (Ba_{1·x}, M²⁺ₓ)FX:yA (where M²⁺ is at least one alkaline earth metal selected from the group consisting of Mg, Ca, Sr, Zn and Cd; X is at least one element selected from the group consisting of Cl, Br and I, A is at least one element selected from the group consisting of Eu, Tb, Ce, Tm, Dy, Pr, Ho, Nd, Yb and Er; x is equal to or greater than 0 and equal to or less than 0.6 and y is equal to or greater than 0 and equal to or less than 0.2) disclosed in U.S. Pat. No. 4,239,968, alkaline earth metal fluorohalide phosphors SrFX:Z (where X is at least one halogen selected from the group consisting of Cl, Br and I; Z is at least one of Eu and Ce) disclosed in Japanese Patent Application Laid Open No. 2-276997, europium activated complex halide phosphors BaFXxNaX′:aEu²⁺ (where each of X or X′ is at least one halogen selected from the group consisting of Cl, Br and I; x is greater than 0 and equal to or less than 2; and y is greater than 0 and equal to or less than 0.2) disclosed in Japanese Patent Application Laid Open No. 59-56479, cerium activated trivalent metal oxyhalide phosphors MOX:xCe (where M is at least one trivalent metal selected from the group consisting of Pr, Nd, Pm, Sm, Eu, Tb, Dy, Ho, Er, Tm, Yb and Bi; X is at least one halogen selected from the group consisting of Br and I; and x is greater than 0 and less than 0.1) disclosed in Japanese Patent Application laid Open No. 58-69281, cerium activated rare earth oxyhalide phosphors LnOX:xCe (where Ln is at least one rare earth element selected from the group consisting of Y, La, Gd and Lu; X is at least one halogen selected from the group consisting of Cl, Br and I; and x is greater than 0 and equal to or less than 0.1) disclosed in U.S. Pat. No. 4,539,137, and europium activated complex halide phosphors M^{II}FXaM^{I}X′bM^{II}X″₂cM^{III}X′″₃xA:yEu²⁺ (where M^{II} is at least one alkaline earth metal selected from the group consisting of Ba, Sr and Ca; M^{I} is at least one alkaline metal selected from the group consisting of Li, Na, K, Rb and Cs; M′^{II} is at least one divalent metal selected from the group consisting of Be and Mg; M^{III} is at least one trivalent metal selected from the group consisting of Al, Ga, In and Ti; A is at least one metal oxide; X is at least one halogen selected from the group consisting of Cl, Br and I; each of X′, X″ and X′″ is at least one halogen selected from the group consisting of F, Cl, Br and I; a is equal to or greater than 0 and equal to or less than 2; b is equal to or greater than 0 and equal to or less than 10⁻²; c is equal to or greater than 0 and equal to or less than 10⁻²; a+b+c is equal to or greater than 10⁻²; x is greater than 0 and equal to or less than 0.5; and y is greater than 0 and equal to or less than 0.2) disclosed in U.S. Pat. No. 4,962,047.

The above and other objects and features of the present invention will become apparent from the following description made with reference to the accompanying drawings.

### BRIEF DESCRIPTION OF THE DRAWINGS

FIG. 1 is a schematic perspective view showing a biochemical analysis unit.

FIG. 2 is a schematic front view showing a spotting device.

FIG. 3 is a schematic longitudinal cross sectional view showing a hybridization reaction vessel.

FIG. 4 is a schematic cross-sectional view showing a stimulable phosphor sheet which is a preferred embodiment of the present invention.

FIG. 5 is a schematic cross-sectional view showing a method for exposing a number of stimulable phosphor layer regions formed in a support of a stimulable phosphor sheet to a radioactive labeling substance contained in a number of spot-like regions formed in a absorptive substrate of a biochemical analysis unit.

FIG. 6 is a schematic view showing a scanner for reading biochemical analysis data in a number of stimulable phosphor layer regions formed in a support of a stimulable phosphor sheet which is a preferred aspect of the present invention.

FIG. 7 is a schematic perspective view showing details in the vicinity of a photomultiplier of a scanner shown in FIG. 6.

FIG. 8 is a schematic cross-sectional view taken along a line A-A in FIG. 7.

FIG. 9 is a schematic cross-sectional view taken along a line B-B in FIG. 7.

FIG. 10 is a schematic cross-sectional view taken along a line CC in FIG. 7.

FIG. 11 is a schematic cross-sectional view taken along a line DD in FIG. 7.

FIG. 12 is a schematic plan view of a scanning mechanism of an optical head.

FIG. 13 is a block diagram of a control system, an input system, a drive system and a detection system of a scanner which is a preferred embodiment of the present invention.

FIG. 14 is a block diagram of a data processing apparatus.

FIG. 15 is a schematic perspective view showing a stimulable phosphor sheet which is another preferred embodiment of the present invention.

FIG. 16 is a schematic perspective view showing a stimulable phosphor sheet which is a further preferred embodiment of the present invention.

### DESCRIPTION OF THE PREFERRED EMBODIMENTS

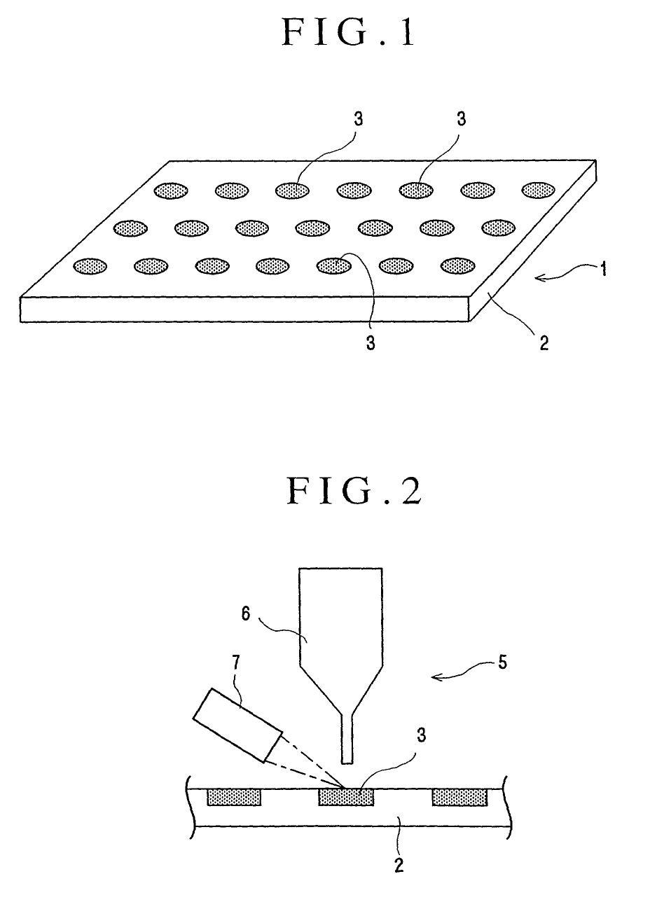

FIG. 1 is a schematic perspective view showing a biochemical analysis unit.

As shown in FIG. 1, a biochemical analysis unit 1 includes a absorptive substrate 2 formed of nylon-6 and a solution containing specific binding substances such as a plurality of cDNAs is spotted on the surface of the absorptive substrate 2 at regular intervals, whereby a number of substantially circular spot-like regions 3 containing specific binding substances are formed in the absorptive substrate 2.

Although not accurately shown in FIG. 1, in this embodiment, substantially circular spot-like regions 3 having a size of about 0.07 cm² are regularly formed in the manner of a matrix of 120 columns×160 lines and, therefore, 19,200 spot-like regions 3 are formed in the absorptive substrate 2.

FIG. 2 is a schematic front view showing a spotting device. As shown in FIG. 2, when biochemical analysis is performed, a solution containing specific binding substances such as a plurality of cDNAs whose sequences are known but differ from each other is spotted using a spotting device 5 onto the surface of the absorptive substrate 2 of the biochemical analysis unit 1, thereby forming a number of the spot-like regions 3.

As shown in FIG. 2, the spotting device 5 includes an injector 6 for ejecting a solution of specific binding substances toward the biochemical analysis unit 1 and a CCD camera 7 and is constituted so that the solution of specific binding substances such as cDNAs is spotted from the injector 6 when the tip end portion of the injector 6 and the center of a region of the absorptive substrate 2 into which the solution containing specific binding substances is to be spotted are determined to coincide with each other as a result of viewing them using the CCD camera, thereby ensuring that the solution of specific binding substances can be accurately spotted on the absorptive substrate 2 of the biochemical analysis unit 1, thereby forming a number of the spot-like regions 3 in a desired manner.

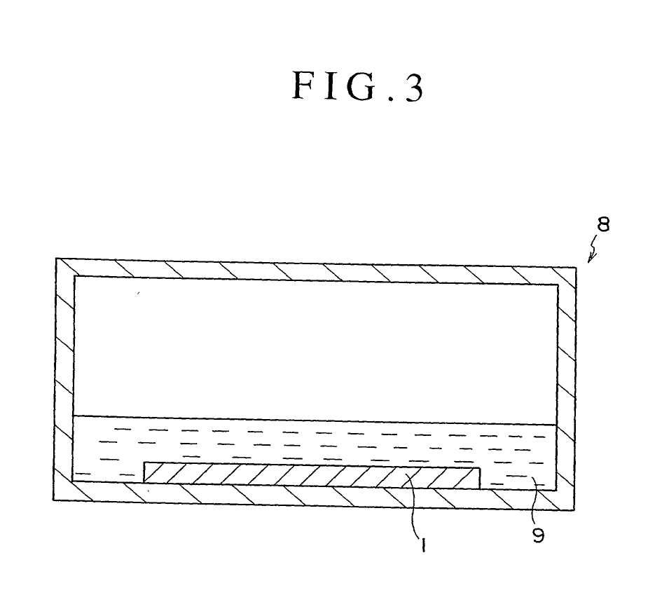

FIG. 3 is a schematic longitudinal cross sectional view showing a hybridization reaction vessel.

As shown in FIG. 3, a hybridization reaction vessel 8 is formed to have a substantially rectangular cross section and accommodates a hybridization solution 9 containing a substance derived from a living organism labeled with a labeling substance as a probe therein.

In this embodiment, a hybridization reaction solution 9 containing a substance derived from a living organism labeled with a radioactive labeling substance is prepared and accommodated in the hybridization reaction vessel 8.

When hybridization is to be performed, the biochemical analysis unit 1 including a number of the spot-like regions 3 formed by regularly spotting the solution containing specific binding substances such as a plurality of cDNAs on the absorptive substrate 2 is accommodated in the hybridization reaction vessel 8.

As a result, specific binding substances spotted in a number of the spot-like regions 3 formed in the absorptive substrate 2 of the biochemical analysis unit 1 can be selectively hybridized with a substance derived from a living organism labeled with a radioactive labeling substance.

In this manner, radiation data of a radioactive labeling substance are recorded in a number of the spot-like regions 3 of the biochemical analysis unit 1.

Radiation data of the radioactive labeling substance recorded in a number of the spot-like regions 3 of the biochemical analysis unit 1 are transferred onto a stimulable phosphor layer of a stimulable phosphor sheet and read by the scanner described later, thereby producing biochemical analysis data.

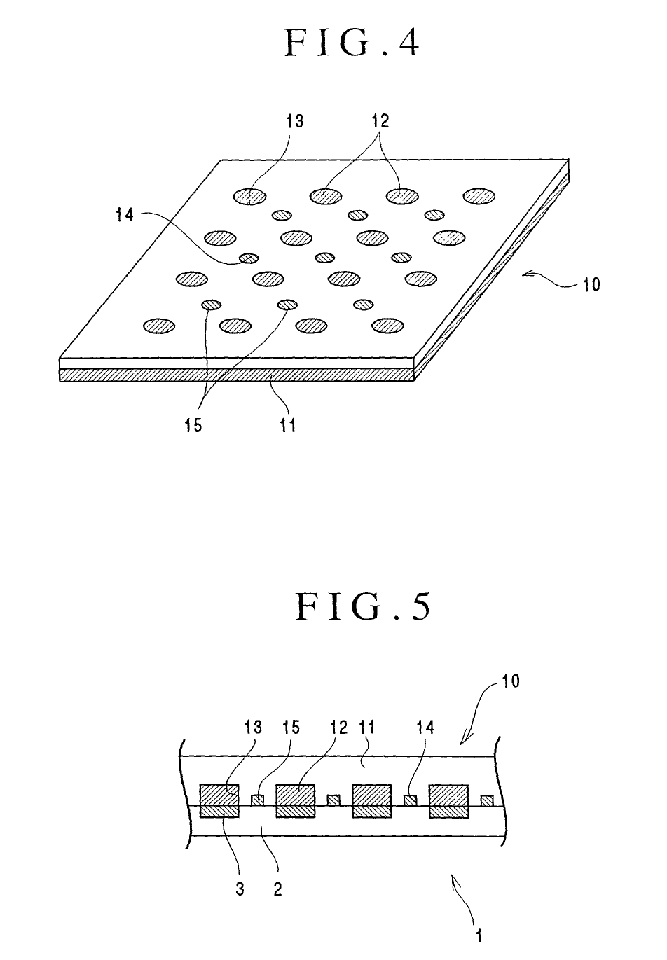

FIG. 4 is a schematic cross-sectional view showing a stimulable phosphor sheet which is a preferred embodiment of the present invention.

As shown in FIG. 4, a stimulable phosphor sheet 10 according to this embodiment includes a support 11 made of stainless steel and regularly formed with a number of substantially circular recesses 13 and a number of recesses 14, a number of stimulable phosphor layer regions 12 formed by embedding stimulable phosphor in a number of the recesses 13 formed in the support 11 and a number of additional stimulable phosphor layer regions 15 formed by embedding stimulable phosphor in a number of the recesses 14 formed in the support 11.

In this embodiment, the area of each of the recesses 14 for forming a number of the additional stimulable phosphor layer regions 15 is smaller than that of each of the recesses 13 for forming a number of the stimulable phosphor layer regions 12 and, therefore, a number of the additional stimulable phosphor layer regions 15 are formed so that the area of each is smaller than that of each of a number of the stimulable phosphor layer regions 12.

Further, in this embodiment, a number of the stimulable phosphor layer regions 12 are formed by embedding stimulable phosphor in the recesses 13 so that the surface of the support 11 and the surfaces of the stimulable phosphor layer regions 12 lie at the same height level and a number of the additional stimulable phosphor layer regions 15 are formed by embedding stimulable phosphor in the recesses 14 so that the surface of the support 11 and the surfaces of the stimulable phosphor layer regions 15 lie at the same height level.

A number of the recesses 13 are formed in the support 11 in the same pattern as that of a number of the spot-like regions 3 formed in the absorptive substrate 2 of the biochemical analysis unit 1 and each of them has the same size as that of the spot-like region 3 formed in the absorptive substrate 2 of the biochemical analysis unit 1.

Therefore, although not accurately shown in FIG. 4, in this embodiment, the substantially circular recesses 13 having a size of about 0.07 cm² are regularly formed in the same pattern as that of a number of the spot-like regions 3 formed in the absorptive substrate 2 of the biochemical analysis unit 1 in the manner of a matrix of 120 columns×160 lines in the support 11 and, therefore, 19,200 recesses 13 are dot-like formed.

As a result, it is possible to superpose the stimulable phosphor sheet 10 and the biochemical analysis unit 1 in such a manner that each of the stimulable phosphor layer regions 12 formed in the support 11 of the stimulable phosphor sheet 10 faces only the corresponding spot-like region 3 formed in the absorptive substrate 2 of the biochemical analysis unit 1, thereby exposing each of the stimulable phosphor layer regions 12 of the stimulable phosphor sheet 10 to a radioactive labeling substance contained in the spot-like region 3 of the biochemical analysis unit 1 the stimulable phosphor layer region 12 faces.

FIG. 5 is a schematic cross-sectional view showing a method for exposing a number of the stimulable phosphor layer regions 12 formed in the support 11 of the stimulable phosphor sheet to a radioactive labeling substance contained in a number of spot-like regions 3 formed in the absorptive substrate 2 of the biochemical analysis unit 1.

As shown in FIG. 5, when a number of the stimulable phosphor layer regions 12 formed in the support 11 of the stimulable phosphor sheet 10 is to be exposed to a radioactive labeling substance contained in a number of spot-like regions 3 formed in the absorptive substrate 2 of the biochemical analysis unit 1, the stimulable phosphor sheet 10 is superposed on the biochemical analysis unit 1 in such a manner that a number of the stimulable phosphor layer regions 12 formed by embedding stimulable phosphor in a number of the recesses 13 formed in the support 11 of the stimulable phosphor sheet 10 face the corresponding spot-like regions 3 formed in the absorptive substrate 2 of the biochemical analysis unit 1.

During the exposure operation, electron beams (β rays) are released from the radioactive labeling substance contained in the spot-like regions 3 of the biochemical analysis unit 1. However, since a number of the stimulable phosphor layer regions 12 of the stimulable phosphor sheet 10 are formed in the support 11 in the same regular pattern as that of a number of the spot-like regions 3 formed in the absorptive substrate 2 of the biochemical analysis unit 1 and the stimulable phosphor sheet 10 is superposed on the biochemical analysis unit 1 in such a manner that each of the stimulable phosphor layer regions 12 faces the corresponding spot-like region 3, electron beams (β rays) released from the radioactive labeling substance contained in the individual spot-like regions 3 formed in the absorptive substrate 2 of the biochemical analysis unit 1 impinge only onto the corresponding stimulable phosphor layer region 12 and since the support 11 of the stimulable phosphor sheet 10 is made of stainless steel capable of attenuating radiation energy, electron beams (β rays) can be prevented from scattering in the support 11 of the stimulable phosphor sheet 10. Therefore, it is possible to selectively expose only the stimulable phosphor layer region 12 each of the spot-like region 3 faces to the electron beams (β rays) released from the radioactive labeling substance contained in each of the spot-like regions 3 formed in the absorptive substrate 2 of the biochemical analysis unit 1 and, on the other hand, it is possible to effectively prevent the electron beams (β rays) released from the radioactive labeling substance contained in a number of the spot-like regions 3 formed in the absorptive substrate 2 of the biochemical analysis unit 1 from entering the additional stimulable phosphor layer regions 15 of the stimulable phosphor sheet 10, thereby preventing the additional stimulable phosphor layer regions 15 from being exposed to the radioactive labeling substance contained in a number of the spot-like regions 3 formed in the absorptive substrate 2 of the biochemical analysis unit 1.

However, since it is extremely difficult to completely wash off radioactive labeling substance adhering to the surface of the biochemical analysis unit 1 where no spot-like region is formed during the hybridization operation, radioactive labeling substance adhering to the surface of the biochemical analysis unit 1 where no spot-like region is formed during the hybridization operation remains after washing the biochemical analysis unit 1, and electron beams (β rays) released from the remaining radioactive labeling substance inevitably enters a number of the stimulable phosphor layer regions 12 formed in the support 11 of the stimulable phosphor sheet 10. Further, ambient radiation also enters a number of the stimulable phosphor layer regions 12 formed in the support 11 of the stimulable phosphor sheet 10. Therefore, biochemical analysis data obtained by scanning a number of the stimulable phosphor layer regions 12 formed in the support 11 of the stimulable phosphor sheet 10 and exposed to the radioactive labeling substance contained in a number of the spot-like regions 3 formed in the absorptive substrate 2 of the biochemical analysis unit 1 with a stimulating ray and photoelectrically detecting stimulated emission released from a number of the stimulable phosphor layer regions 12 formed in the support 11 of the stimulable phosphor sheet 10 inevitably contain data corresponding to background noise caused by electron beams (β rays) released from the radioactive labeling substance adhering to the surface of the biochemical analysis unit 1 where no spot-like region is formed during the hybridization operation and remaining after the washing operation, ambient radiation and the like entering a number of the stimulable phosphor layer regions 12 formed in the support 11 of the stimulable phosphor sheet 10.

On the other hand, since electron beams (β rays) released from the radioactive labeling substance contained in a number of the spot-like regions 3 formed in the absorptive substrate 2 of the biochemical analysis unit 1 do not enter a number of the additional stimulable phosphor layer regions 15 of the stimulable phosphor sheet 10 and a number of the additional stimulable phosphor layer regions 15 of the stimulable phosphor sheet 10 are prevented from being exposed to the electron beams (β rays) released from the radioactive labeling substance contained in a number of the spot-like regions 3 formed in the absorptive substrate 2 of the biochemical analysis unit 1, only electron beams (β rays) released from radioactive labeling substance adhering to the surface of the biochemical analysis unit 1 where no spot-like region is formed during the hybridization operation and remaining after the washing operation, ambient radiation and the like enter a number of the additional stimulable phosphor layer regions 15 of the stimulable phosphor sheet 10 and a number of the additional stimulable phosphor layer regions 15 of the stimulable phosphor sheet 10 are exposed to only the electron beams (β rays) released from radioactive labeling substance adhering to the surface of the biochemical analysis unit 1 where no spot-like region is formed during the hybridization operation and remaining after the washing operation, ambient radiation and the like. Therefore, data obtained by scanning a number of the additional stimulable phosphor layer regions 15 of the stimulable phosphor sheet 10 with a stimulating ray and photoelectrically detecting stimulated emission released from a number of the additional stimulable phosphor layer regions 15 of the stimulable phosphor sheet 10 correspond to background noise.

In this manner, radiation data of the radioactive labeling substance are recorded in a number of the stimulable phosphor layer regions 12 formed in the support 11 of the stimulable phosphor sheet 10.

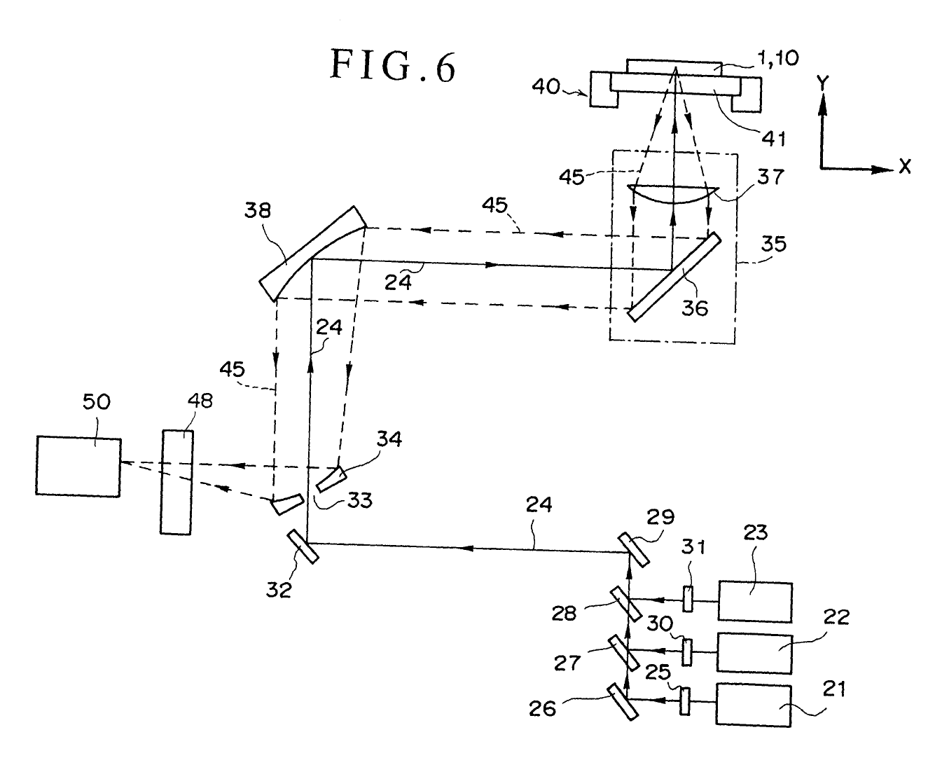

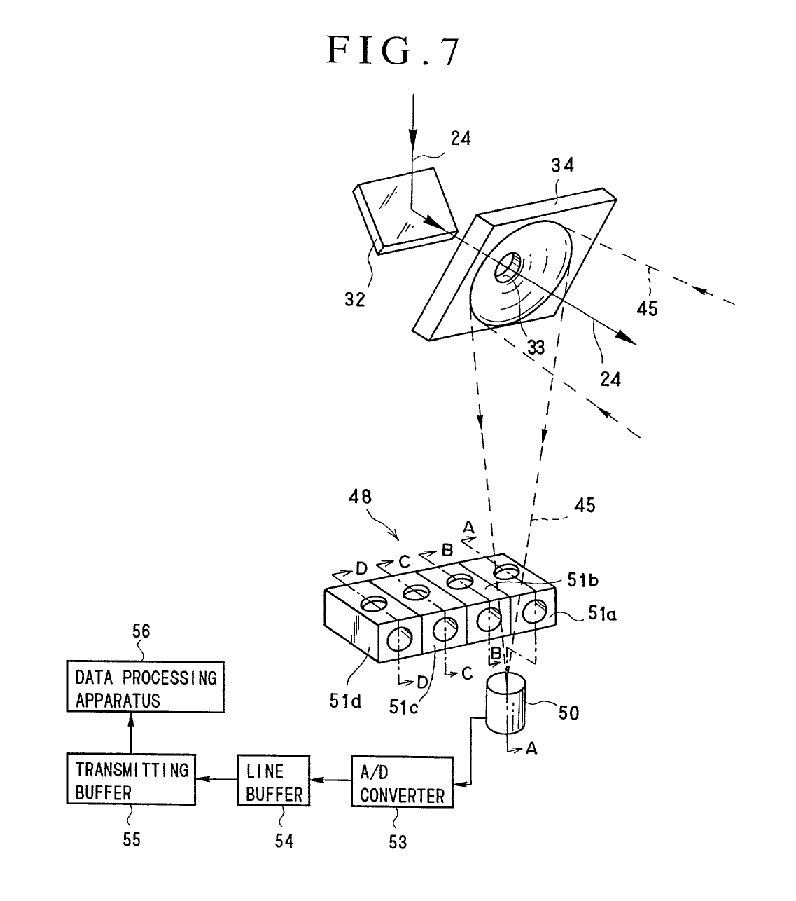

FIG. 6 is a schematic view showing a scanner for reading biochemical analysis data in a number of the stimulable phosphor layer regions 12 formed in the support 11 of the stimulable phosphor sheet 10 which is a preferred aspect of the present invention and FIG. 7 is a schematic perspective view showing details in the vicinity of a photomultiplier of the scanner.

The scanner shown in FIGS. 6 and 7 is constituted so as to read radiation data of a radioactive labeling substance recorded in a number of the stimulable phosphor layer regions 12 formed in the support 11 of the stimulable phosphor sheet 10 and fluorescence data of a fluorescent substance such as a fluorescent dye recorded in a gel support or a transfer support and includes a first laser stimulating ray source 21 for emitting a laser beam having a wavelength of 640 nm, a second laser stimulating ray source 22 for emitting a laser beam having a wavelength of 532 nm and a third laser stimulating ray source 23 for emitting a laser beam having a wavelength of 473 nm.

In this embodiment, the first laser stimulating ray source 21 is constituted by a semiconductor laser beam source and the second laser stimulating ray source 22 and the third laser stimulating ray source 23 are constituted by a second harmonic generation element.

A laser beam 24 emitted from the first laser stimulating source 21 passes through a collimator lens 25, thereby being made a parallel beam, and is reflected by a mirror 26. A first dichroic mirror 27 for transmitting light having a wavelength of 640 nm but reflecting light having a wavelength of 532 nm and a second dichroic mirror 28 for transmitting light having a wavelength equal to and longer than 532 nm but reflecting light having a wavelength of 473 nm are provided in the optical path of the laser beam 24 emitted from the first laser stimulating ray source 21. The laser beam 24 emitted from the first laser stimulating ray source 21 and reflected by the mirror 26 passes through the first dichroic mirror 27 and the second dichroic mirror 28 and advances to a mirror 29.

On the other hand, the laser beam 24 emitted from the second laser stimulating ray source 22 passes through a collimator lens 30, thereby being made a parallel beam, and is reflected by the first dichroic mirror 27, thereby changing its direction by 90 degrees. The laser beam 24 then passes through the second dichroic mirror 28 and advances to the mirror 29.

Further, the laser beam 24 emitted from the third laser stimulating ray source 23 passes through a collimator lens 31, thereby being made a parallel beam, and is reflected by the second dichroic mirror 28, thereby changing its direction by 90 degrees. The laser beam 24 then advances to the mirror 29.

The laser beam 24 advancing to the mirror 29 is reflected by the mirror 29 and advances to a mirror 32 to be reflected thereby.

A perforated mirror 34 formed with a hole 33 at the center portion thereof is provided in the optical path of the laser beam 24 reflected by the mirror 32. The laser beam 24 reflected by the mirror 32 passes through the hole 33 of the perforated mirror 34 and advances to a concave mirror 38.

The laser beam 24 advancing to the concave mirror 38 is reflected by the concave mirror 38 and enters an optical head 35.

The optical head 35 includes a mirror 36 and an aspherical lens 37. The laser beam 24 entering the optical head 35 is reflected by the mirror 36 and condensed by the aspherical lens 37 onto the stimulable phosphor sheet 10, or a gel support or a transfer support placed on the glass plate 41 of a stage 40.

When the laser beam 24 impinges on the stimulable phosphor layer region 12 formed in the support 11 of the stimulable phosphor sheet 10, stimulable phosphor contained in the stimulable phosphor layer region 12 is excited, thereby releasing stimulated emission 45. On the other hand, when the laser beam 24 impinges on the gel support or the transfer support, a fluorescent dye or the like contained therein is excited, thereby releasing fluorescence emission 45.

The stimulated emission 45 released from the stimulable phosphor layer region 12 of the stimulable phosphor 10 or the fluorescence emission 45 released from the gel support or the transfer supportgel support or the transfer support is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 or the fluorescence emission 45 advancing to the concave mirror 38 is reflected by the concave mirror 38 and advances to the perforated mirror 34.

As shown in FIG. 7, the stimulated emission 45 or the fluorescence emission 45 advancing to the perforated mirror 34 is reflected downward by the perforated mirror 34 formed as a concave mirror and advances to a filter unit 48, whereby light having a predetermined wavelength is cut. The stimulated emission 45 or the fluorescence emission 45 then impinges on a photomultiplier 50, thereby being photoelectrically detected.

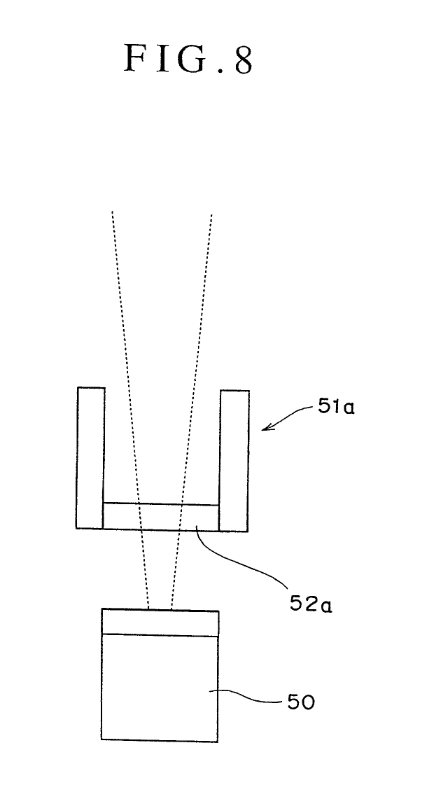

As shown in FIG. 8, the filter unit 48 is provided with four filter members 51a, 51b, 51c and 51d and is constituted to be laterally movable in FIG. 7 by a motor (not shown).

FIG. 8 is a schematic cross-sectional view taken along a line A-A in FIG. 7.

As shown in FIG. 8, the filter member 51a includes a filter 52a and the filter 52a is used for reading fluorescence emission 45 by stimulating a fluorescent substance such as a fluorescent dye contained in a gel support or a transfer support using the first laser stimulating ray source 21 and has a property of cutting off light having a wavelength of 640 nm but transmitting light having a wavelength longer than 640 nm.

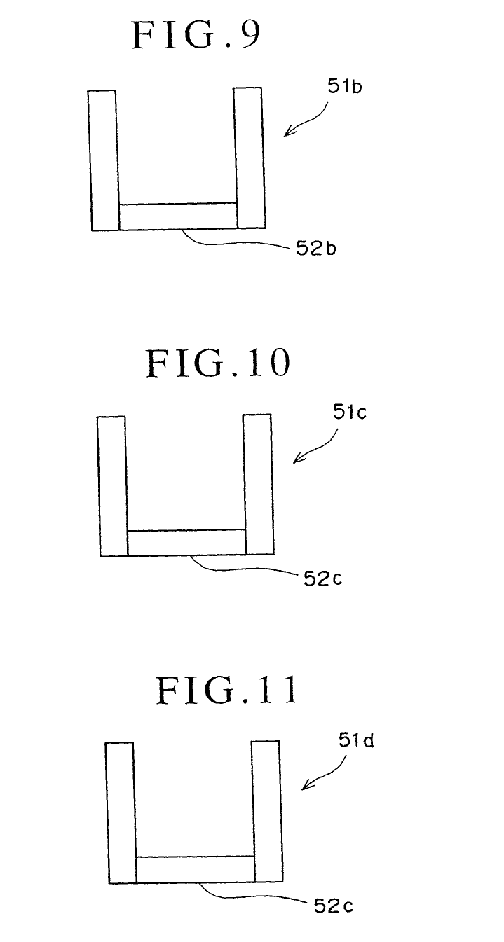

FIG. 9 is a schematic cross-sectional view taken along a line B-B in FIG. 7.

As shown in FIG. 9, the filter member 51b includes a filter 52b and the filter 52b is used for reading fluorescence emission 45 by stimulating a fluorescent substance such as a fluorescent dye contained in a gel support or a transfer support using the second laser stimulating ray source 22 and has a property of cutting off light having a wavelength of 532 nm but transmitting light having a wavelength longer than 532 nm.

FIG. 10 is a schematic cross-sectional view taken along a line CC in FIG. 7.

As shown in FIG. 10, the filter member 51c includes a filter 52c and the filter 52c is used for reading fluorescence emission 45 by stimulating a fluorescent substance such as a fluorescent dye contained in a gel support or a transfer support using the third laser stimulating ray source 23 and has a property of cutting off light having a wavelength of 473 nm but transmitting light having a wavelength longer than 473 nm.

FIG. 11 is a schematic cross-sectional view taken along a line DD in FIG. 7.

As shown in FIG. 11, the filter member 51d includes a filter 52d and the filter 52d is used for reading stimulated emission released from stimulable phosphor contained in the stimulable phosphor layer 12 formed in the support 11 of the stimulable phosphor sheet 10 upon being stimulated using the first laser stimulating ray source 1 and has a property of transmitting only light having a wavelength corresponding to that of stimulated emission emitted from stimulable phosphor but cutting off light having a wavelength of 640 nm.

Therefore, in accordance with the kind of a stimulating ray source to be used, one of these filter members 51a, 51b, 51c, 51d is selectively positioned in front of the photomultiplier 50, thereby enabling the photomultiplier 50 to photoelectrically detect only light to be detected. The analog data produced by photoelectrically detecting light with the photomultiplier 50 are converted with a scale factor suitable for the signal fluctuation width by an A/D converter 53 into digital data and the digital data are fed to a line buffer 54.

The line buffer 54 is constituted so as to temporarily store digital data corresponding to one scanning line. When the digital data corresponding to one scanning line have been stored in the line buffer 54 in the above described manner, the line buffer 54 outputs the digital data to a transmitting buffer 55 whose capacity is greater than that of the line buffer 54 and when the transmitting buffer 55 has stored a predetermined amount of the digital data, it outputs the digital data to a data processing apparatus 56.

Although not shown in FIG. 6, the optical head 35 is constituted to be movable by a scanning mechanism in a main scanning direction indicated by an arrow X and a sub-scanning direction indicated by an arrow Y in FIG. 7 so that all of the stimulable phosphor layer regions 12 formed in the support 11 of the stimulable phosphor sheet 10 or the whole surface of a gel support or a transfer support can be scanned by the laser beam 24.

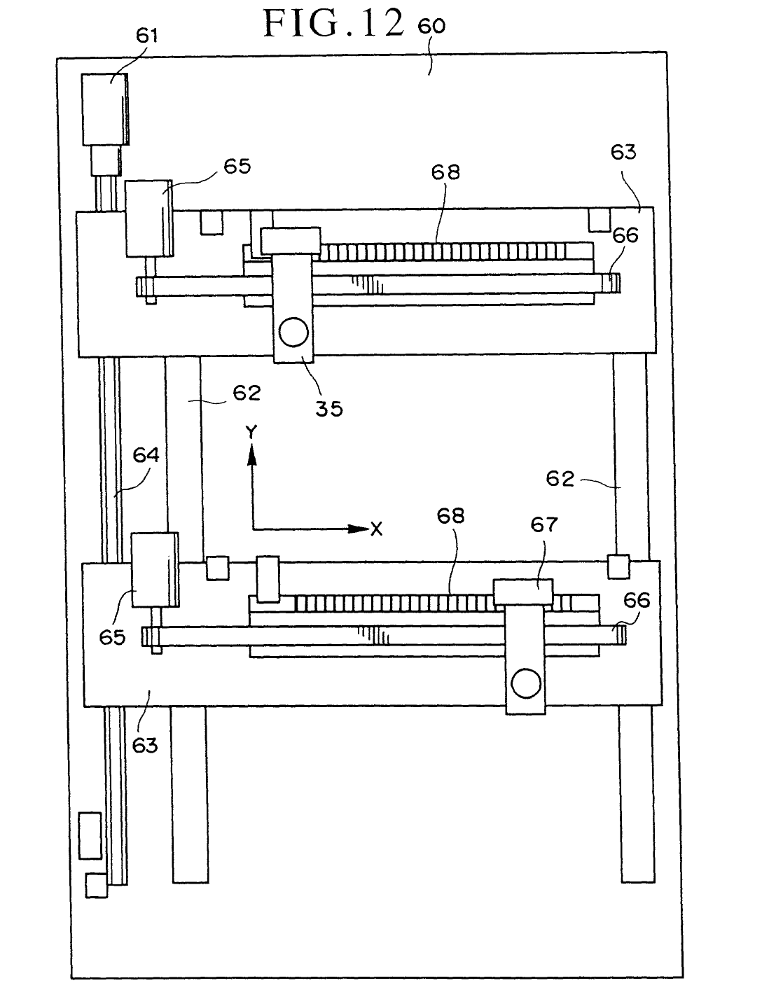

FIG. 12 is a schematic plan view showing the scanning mechanism of the optical head 35.

In FIG. 12, optical systems other than the optical head 35 and the paths of the laser beam 24 and stimulated emission 45 or fluorescence emission 45 are omitted for simplification.

As shown in FIG. 12, the scanning mechanism of the optical head 35 includes a base plate 60, and a sub-scanning pulse motor 61 and a pair of rails 62, 62 are fixed on the base plate 60. A movable base plate 63 is further provided so as to be movable in the sub-scanning direction indicated by an arrow Y in FIG. 12.

The movable base plate 63 is formed with a threaded hole (not shown) and a threaded rod 64 rotated by the sub-scanning pulse motor 61 is engaged with the inside of the hole.

A main scanning pulse motor 65 is provided on the movable base plate 63. The main pulse stepping motor 65 is adapted for driving an endless belt 66. The optical head 35 is fixed to the endless belt 66 and when the endless belt 66 is driven by the main scanning stepping motor 65, the optical head 35 is moved in the main scanning direction indicated by an arrow X in FIG. 12.

In FIG. 12, the reference numeral 67 designates a linear encoder for detecting the position of the optical head 35 in the main scanning direction and the reference numeral 68 designates slits of the linear encoder 67.

Therefore, the optical head 35 is moved in the main scanning direction indicated by the arrow X and the sub-scanning direction indicated by the arrow Y in FIG. 12 by driving the endless belt 66 in the main scanning direction by the main scanning pulse motor 65 and intermittently moving the movable base plate 63 in the sub-scanning direction by the sub-scanning pulse motor 61, thereby scanning all of the stimulable phosphor layer regions 12 formed on the support 11 of the stimulable phosphor sheet 10 or the whole surface of a gel support or a transfer support with the laser beam 24.

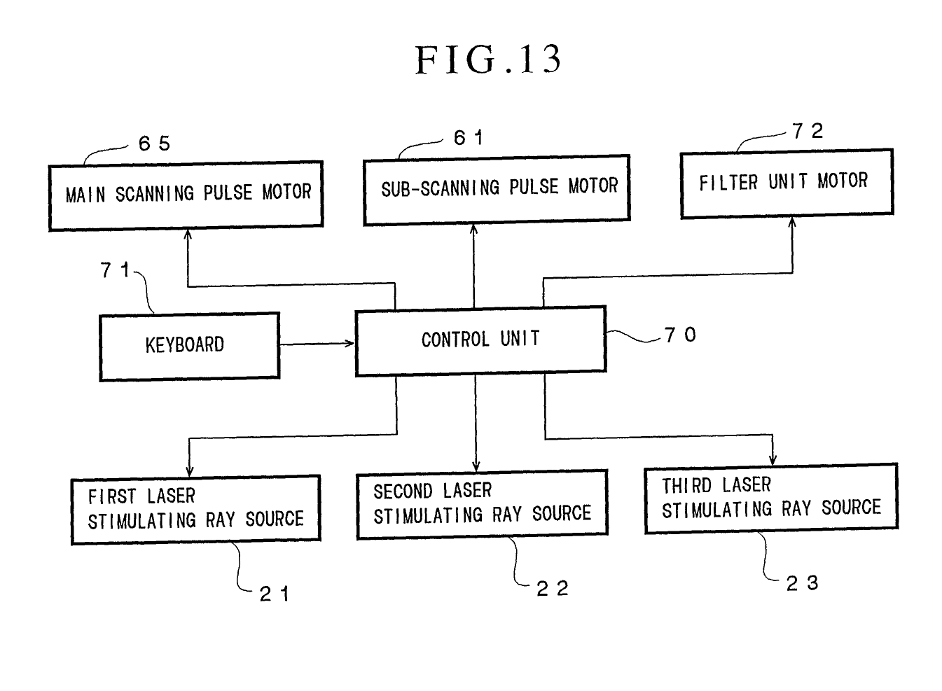

FIG. 13 is a block diagram of a control system, an input system and a drive system of the scanner shown in FIG. 6.

As shown in FIG. 13, the control system of the scanner includes a control unit 70 for controlling the overall operation of the scanner and the input system of the scanner includes a keyboard 71 which can be operated by a user and through which various instruction signals can be input.

As shown in FIG. 13, the drive system of the scanner includes the main scanning pulse motor 65 for moving the optical head 35 in the main scanning direction, the sub-scanning pulse motor 61 for moving the optical head 35 in the sub-scanning direction and a filter unit motor 72 for moving the filter unit 48 provided with the four filter members 51a, 51b, 51c and 51d.

The control unit 70 is adapted for selectively outputting a drive signal to the first laser stimulating ray source 21, the second laser stimulating ray source 22 or the third laser stimulating ray source 23 and outputting a drive signal to the filter unit motor 72.

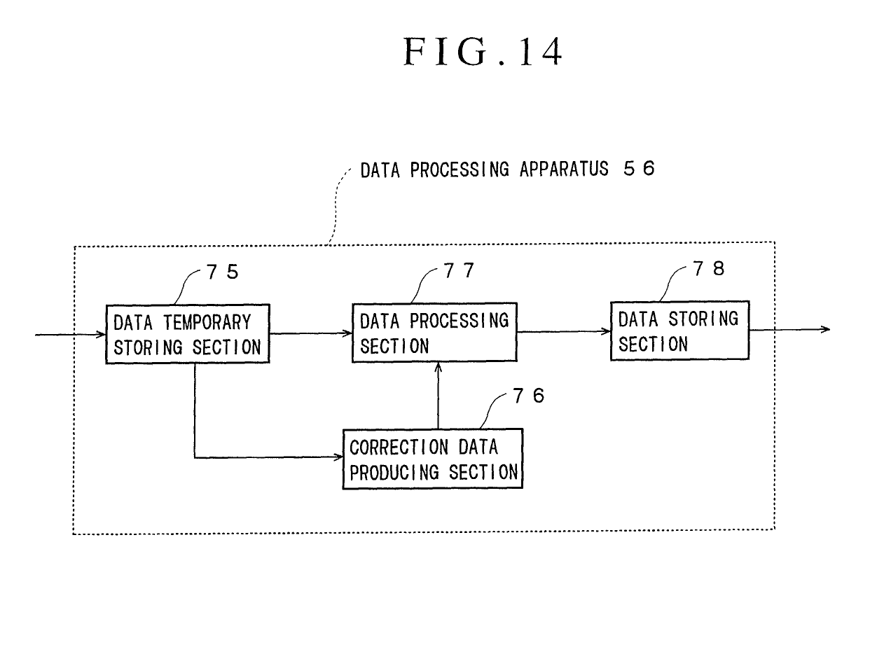

FIG. 14 is a block diagram of the data processing apparatus 56. As shown in FIG. 14, the data processing apparatus 56 includes a data temporary storing section 75 for receiving digital data temporarily stored in the transmitting buffer 55 and temporarily storing them, a correction data producing section 76 for producing background noise correction data based on digital data stored in the data temporary storing section 75, a data processing section 77 for effecting predetermined data processing on digital data, for example, reading digital data stored in the data temporary storing section 75 and effecting background noise correction on them based on background noise correction data produced by the correction data producing section 76, and a data storing section 78 for storing digital data subjected to data processing.

The thus constituted scanner reads radiation data recorded in a stimulable phosphor sheet 10 by exposing a number of the stimulable phosphor layer regions 12 to a radioactive labeling substance contained in a number of the spot-like regions 3 formed in the absorptive substrate 2 of the biochemical analysis unit 1 and produces biochemical analysis data in the following manner.

A stimulable phosphor sheet 10 is first set on the glass plate 41 of the stage 40 by a user.

An instruction signal indicating that radiation data recorded in a number of the stimulable phosphor layer regions 12 formed in the support 11 of the stimulable phosphor sheet 10 are to be read is then input through the keyboard 71 The instruction signal input through the keyboard 71 is input to the control unit 70 and when the control unit 70 receives the instruction signal, it outputs a drive signal to the filter unit motor 72 in accordance with the instruction signal, thereby moving the filter unit 48 so as to locate the filter member 51d provided with the filter 52d having a property of transmitting only light having a wavelength corresponding to that of stimulated emission emitted from stimulable phosphor but cutting off light having a wavelength of 640 nm in the optical path of stimulated emission 45.

The control unit 70 then outputs a drive signal to the first laser stimulating ray source 21 to activate it, thereby causing it to emit a laser beam 24 having a wavelength of 640 nm.

The laser beam 24 emitted from the first laser stimulating ray source 21 is made a parallel beam by the collimator lens 25 and advances to the mirror 26 to be reflected thereby.

The laser beam 24 reflected by the mirror 26 passes through the first dichroic mirror 27 and the second dichroic mirror 28 and advances to the mirror 29.

The laser beam 24 advancing to the mirror 29 is reflected by the mirror 29 and further advances to a mirror 32 to be reflected thereby.

The laser beam 24 reflected by the mirror 32 passes through the hole 33 of the perforated mirror 34 and advances to the concave mirror 38.

The laser beam 24 advancing to the concave mirror 38 is reflected thereby and enters the optical head 35.

The laser beam 24 entering the optical head 35 is reflected by the mirror 36 and condensed by the aspherical lens 37 onto a stimulable phosphor layer region 12 formed in the support 11 of the stimulable phosphor sheet 10 placed on the glass plate 41 of the stage 40.

In this embodiment, since a number of the stimulable phosphor layer regions 12 of the stimulable phosphor sheet 10 are formed spaced apart from each other in the support 11 made of stainless steel capable of attenuating radiation energy, it is possible to efficiently prevent a laser beam 24 entering the stimulable phosphor layer region 12 from scattering and stimulating stimulable phosphor contained in stimulable phosphor layer regions 12.

When the laser beam 24 impinges on the stimulable phosphor layer region 12 formed in the support 11 of stimulable phosphor sheet 10, stimulable phosphor contained in the stimulable phosphor layer region 12 is excited by the laser beam 24 and stimulated emission is released from the stimulable phosphor.

The stimulated emission 45 released from the stimulable phosphor contained in the stimulable phosphor layer region 12 of the stimulable phosphor sheet 10 is condensed by the aspherical lens 37 provided in the optical head 35 and reflected by the mirror 36 on the side of an optical path of the laser beam 24, thereby being made a parallel beam to advance to the concave mirror 38.

The stimulated emission 45 advancing to the concave mirror 38 is reflected by the concave mirror 38 and advances to the perforated mirror 34.

As shown in FIG. 7, the stimulated emission 45 advancing to the perforated mirror 34 is reflected downward by the perforated mirror 34 formed as a concave mirror and advances to the filter 52d of the filter unit 48.

Since the filter 52d has a property of transmitting only light having a wavelength corresponding to that of stimulated emission emitted from stimulable phosphor but cutting off light having a wavelength of 640 nm, light having a wavelength of 640 nm corresponding to that of the stimulating ray is cut off by the filter 52d and only light having a wavelength corresponding to that of stimulated emission passes through the filter 52d to be photoelectrically detected by the photomultiplier 50. As described above, since the optical head 35 is moved on the base plate 63 in the main scanning direction indicated by the arrow X in FIG. 12 by the main scanning pulse motor 65 mounted on the base plate 63 and the base plate 63 is moved in the sub-scanning direction indicated by the arrow Y in FIG. 12 by the sub-scanning pulse motor 61, all of the stimulable phosphor layer regions 12 formed in the support 11 of the stimulable phosphor sheet 10 are scanned by the laser beam 24. Therefore, the photomultiplier 50 can read radiation data of a radioactive labeling substance recorded in a number of the stimulable phosphor layer regions 12 of the stimulable phosphor sheet 10 by photoelectrically detecting the stimulated emission 45 released from stimulable phosphor contained in the stimulable phosphor layer regions 12 of the stimulable phosphor sheet 10 and produce analog data for biochemical analysis.

Since the stimulable phosphor sheet 10 includes a number of the additional stimulable phosphor layer regions 15 formed by embedding stimulable phosphor in a number of the recesses 14 formed in the support 11 between a number of the stimulable phosphor layer regions 12 and a number of the additional stimulable phosphor layer regions 15 are exposed to electron beams (β rays) released from radioactive labeling substance adhering to the surface of the substrate 2 of the biochemical analysis unit 1 during hybridization and remaining even after washing, ambient radiation and the like and stores radiation energy, when the stimulable phosphor sheet 10 is scanned with the laser beam 24, stimulable phosphor contained in a number of the additional stimulable phosphor layer regions 15 is excited by the laser beam 24 to release stimulated emission 45 and the stimulated emission 45 released from a number of the additional stimulable phosphor layer regions 15 is photoelectrically detected by the photomultiplier 50 similarly to stimulated emission 45 released from a number of the stimulable phosphor layer regions 12.

Therefore, the analog data produced by scanning all of the stimulable phosphor layer regions 12 formed in the support 11 of the stimulable phosphor sheet 10 contain analog data obtained by detecting stimulated emission 45 released from a number of the additional stimulable phosphor layer regions 15 formed in the support 11 of the stimulable phosphor sheet 10.

The analog data produced by photoelectrically detecting light with the photomultiplier 50 are converted with a scale factor suitable for the signal fluctuation width by an A/D converter 53 into digital data and the digital data are fed to a line buffer 54.

When the digital data corresponding to one scanning line have been stored in the line buffer 54 in the above described manner, the line buffer 54 outputs the digital data to a transmitting buffer 55 whose capacity is greater than that of the line buffer 54 and when the transmitting buffer 55 has stored a predetermined amount of the digital data, it outputs the digital data to the data processing apparatus 56.

The digital data output to the data processing apparatus 56 are temporarily stored in the data temporary storing section 75. The digital data temporarily stored in the data temporary storing section 75 are output to the correction data producing section 76 as well as the data processing section 77.

As described above, a number of the additional stimulable phosphor layer regions 15 formed in the support 11 of the stimulable phosphor sheet 10 are exposed to only electron beams (β rays) released from radioactive labeling substance adhering to the surface of the substrate 2 of the biochemical analysis unit 1 during hybridization and remaining even after washing, ambient radiation and the like and are not exposed to electron beams (β rays) released from the radioactive labeling substance selectively contained in a number of the stimulable phosphor layer regions 12 formed in the support 11 of the stimulable phosphor sheet 10. Therefore, since digital data obtained by scanning a number of the additional stimulable phosphor layer regions 15 formed in the support 11 of the stimulable phosphor sheet 10 with the laser beam 24 and photoelectrically detecting stimulated emission 45 released from a number of the additional stimulable phosphor layer regions 15 correspond to background noise, the correction data producing section 76 produces background noise correction data from the digital data obtained by photoelectrically detecting stimulated emission 45 released from a number of the additional stimulable phosphor layer regions 15 based on the digital data input from the data temporary storing section 75 and outputs the thus produced background noise correction data to the data processing section 77.

The data processing section 77 subtracts the background noise correction data input from the correction data producing section 76 from the digital data input from the data temporary storing section 75, thereby eliminating background noise and further effects necessary data processing on the digital data. The data processing section 77 then stores the data-processed digital data in the data storing section 78 and erases the digital data stored in the data temporary storing section 75.

Quantitative analysis is performed based on the digital data in which background noise has been eliminated in this manner and which have been further subjected to data processing as occasion demands and stored in the data storing section 78.

On the other hand, when fluorescence data such as electrophoresis data of denatured DNA fragments labeled with a fluorescent substance such as a fluorescent dye recorded in a gel support or a transfer support are to be read to produce biochemical analysis data, a gel support or a transfer support is first set on the glass plate 41 of the stage 40 by a user.

A fluorescent substance identification signal for identifying the kind of fluorescent substance that is the labeling substance is then input through the keyboard 71 by the user together with an instruction signal indicating that fluorescence data are to be read.

When the kind of fluorescent substance is input by the user through the keyboard 71, the control unit 70 selects a laser stimulating ray source for emitting a laser beam 24 of a wavelength capable of efficiently stimulating the identified fluorescent substance from among the first laser stimulating ray source 21, the second laser stimulating ray source 22 and the third laser stimulating ray source 23 and selects the filter member for cutting light having a wavelength of the laser beam 24 to be used for stimulating the input fluorescent substance and transmitting light having a longer wavelength than that of the laser beam to be used for stimulation from among the three filter members 51a, 51b and 51c.

The whole surface of the gel support or the transfer support is then scanned with the laser beam 24 and fluorescence emission is photoelectrically detected by the photomultiplier 50 to produce analog data. The analog data are digitized by the A/D converter, thereby producing biochemical analysis data.

According to the above described embodiment, when a number of the stimulable phosphor layer regions 12 formed in the support 11 of the stimulable phosphor sheet 10 are to be exposed to a radioactive labeling substance selectively contained in a number of the spot-like regions 3 formed in the absorptive substrate 2 of the biochemical analysis unit 1, although electron beams (β rays) are released from the radioactive labeling substance selectively contained in a number of the spot-like regions 3 of the biochemical analysis unit 1, since a number of the stimulable phosphor layer regions 12 of the stimulable phosphor sheet 10 are formed in the support 11 in the same regular pattern as that of a number of the spot-like regions 3 formed in the absorptive substrate 2 of the biochemical analysis unit 1 and the stimulable phosphor sheet 10 is superposed on the biochemical analysis unit 1 in such a manner that each of the stimulable phosphor layer regions 12 faces the corresponding spot-like region 3, electron beams (β rays) released from the radioactive labeling substance contained in the individual spot-like regions 3 formed in the absorptive substrate 2 of the biochemical analysis unit 1 impinge only onto the corresponding stimulable phosphor layer region 12 and since the support 11 of the stimulable phosphor sheet 10 is made of stainless steel capable of attenuating radiation energy, electron beams (β rays) can be prevented from scattering in the support 11 of the stimulable phosphor sheet 10. Therefore, since it is possible to selectively expose only the stimulable phosphor layer region 12 each of the spot-like region 3 faces to the electron beams (β rays) released from the radioactive labeling substance contained in each of the spot-like regions 3 formed in the absorptive substrate 2 of the biochemical analysis unit 1, it is possible to produce biochemical analysis data having excellent quantitative characteristics with high resolution by scanning a number of the exposed stimulable phosphor layer regions 12 with the laser beam 24 and photoelectrically detecting stimulated emission 45 released from a number of the stimulable phosphor layer regions 12.

However, since it is extremely difficult to completely wash off radioactive labeling substance adhering to the surface of the biochemical analysis unit 1 where no spot-like region is formed during the hybridization operation, even when a number of the stimulable phosphor layer regions 12 are formed in the support 11 of the stimulable phosphor sheet 10, radioactive labeling substance adhering to the surface of the biochemical analysis unit 1 where no spot-like region is formed during the hybridization operation remains after washing the biochemical analysis unit 1 and electron beams (β rays) released from the remaining radioactive labeling substance inevitably enter a number of the stimulable phosphor layer regions 12 formed in the support 11 of the stimulable phosphor sheet 10. Further, ambient radiation also enters a number of the stimulable phosphor layer regions 12 formed in the support 11 of the stimulable phosphor sheet 10. Therefore, biochemical analysis data obtained by scanning a number of the stimulable phosphor layer regions 12 formed in the support 11 of the stimulable phosphor sheet 10 and exposed to the radioactive labeling substance selectively contained in a number of the spot-like regions 3 formed in the absorptive substrate 2 of the biochemical analysis unit 1 with the laser beam 24 and photoelectrically detecting stimulated emission released from a number of the stimulable phosphor layer regions 12 formed in the support 11 of the stimulable phosphor sheet 10 inevitably contain background noise caused by electron beams (β rays) released from radioactive labeling substance adhering to the surface of the biochemical analysis unit 1 where no spot-like region is formed during the hybridization operation and remaining after the washing operation, ambient radiation and the like entering a number of the stimulable phosphor layer regions 12 formed in the support 11 of the stimulable phosphor sheet 10.

Nevertheless, according to the above described embodiment, since it is possible not only to selectively expose each of number of the stimulable phosphor layer regions 12 formed in the support 11 of the stimulable phosphor sheet 10 to the radioactive labeling substance contained in the corresponding spot-like region 3 formed in the absorptive substrate 2 of the biochemical analysis unit 1 but also to effectively prevent electron beams (β rays) released from the radioactive labeling substance selectively contained in a number of the spot-like regions 3 formed in the absorptive substrate 2 of the biochemical analysis unit 1 from impinging onto a number of the additional stimulable phosphor layer regions 15 formed in the support 11 of the stimulable phosphor sheet 10, thereby preventing the additional stimulable phosphor layer regions 15 from being exposed to the radioactive labeling substance contained in a number of the spot-like regions 3 formed in the absorptive substrate 2 of the biochemical analysis unit 1, only electron beams (β rays) released from radioactive labeling substance adhering to the surface of the biochemical analysis unit 1 where no spot-like region is formed during the hybridization operation and remaining after the washing operation, ambient radiation and the like enter a number of the additional stimulable phosphor layer regions 15 formed in the support 11 of the stimulable phosphor sheet 10 and a number of the additional stimulable phosphor layer regions 15 formed in the support 11 of the stimulable phosphor sheet 10 are exposed to only the electron beams (β rays) released from radioactive labeling substance adhering to the surface of the biochemical analysis unit 1 where no spot-like region is formed during the hybridization operation and remaining after the washing operation, ambient radiation and the like. As a result, digital data obtained by scanning a number of the additional stimulable phosphor layer regions 15 of the stimulable phosphor sheet 10 with a stimulating ray and photoelectrically detecting stimulated emission released from a number of the additional stimulable phosphor layer regions 15 of the stimulable phosphor sheet 10 correspond to background noise.

Therefore, according to this embodiment, since the correction data producing section 76 of the data processing apparatus 56 produces background noise correction data from digital data produced by photoelectrically detecting stimulated emission 45 released from a number of the additional stimulable phosphor layer regions 15 formed in the support 11 of the stimulable phosphor sheet 10 and the data processing section 77 subtracts the background noise correction data produced by the correction data producing section 76 from digital data produced by scanning the whole surface of the stimulable phosphor sheet 10 with the laser beam 24, thereby eliminating the background noise, it is possible to produce biochemical analysis data free of background noise with high accuracy.

Furthermore, according to this embodiment, since a number of the additional stimulable phosphor layer regions 15 of the stimulable phosphor sheet 10 are formed by embedding stimulable phosphor in a number of the recesses 14 regularly formed in the support 11 between a number of the stimulable phosphor layer regions 12, even if the background noise differs between different positions on the surface of the stimulable phosphor sheet 10, it is possible to produce biochemical analysis data free of background noise with high accuracy.

FIG. 15 is a schematic perspective view showing a stimulable phosphor sheet which is another preferred embodiment of the present invention.

As shown in FIG. 15, a stimulable phosphor sheet 80 according to this embodiment includes a support 81 made of silicon nitride, a number of stimulable phosphor layer regions 82 formed by embedding stimulable phosphor in a number of through-holes 83 formed spaced apart from each other in the support 81, and stripe shaped additional stimulable phosphor layer regions 85 formed by embedding stimulable phosphor in two grooves 84 formed in the support 81 between a number of the stimulable phosphor layer regions 82 so as to be perpendicular to each other.

A number of the stimulable phosphor layer regions 82 are formed in the support 81 in the same regular pattern as that of a number of the spot-like regions 3 formed in the absorptive substrate 2 of the biochemical analysis unit 1 and the stimulable phosphor sheet 80 is constituted so that each of the stimulable phosphor layer regions 82 faces only the corresponding spot-like region 3 formed in the absorptive substrate 2 of the biochemical analysis unit 1.

In this embodiment, when a number of the stimulable phosphor layer regions 82 formed in the support 81 of the stimulable phosphor sheet 80 are to be exposed to a radioactive labeling substance selectively contained in a number of the spot-like regions 3 formed in the absorptive substrate 2 of the biochemical analysis unit 1, the stimulable phosphor sheet 80 is superposed on the biochemical analysis unit 1 in such a manner that each of a number of the stimulable phosphor layer regions 82 formed in the support 81 of the stimulable phosphor sheet 80 in the same regular pattern as that of a number of the spot-like regions 3 formed in the absorptive substrate 2 of the biochemical analysis unit 1 faces the corresponding spot-like region 3 formed in the absorptive substrate 2 of the biochemical analysis unit 1.

Therefore, electron beams (β rays) released from the radioactive labeling substance contained in the individual spot-like regions 3 formed in the absorptive substrate 2 of the biochemical analysis unit 1 impinge only onto the corresponding stimulable phosphor layer region 82 and since the support 81 of the stimulable phosphor sheet 80 is made of silicon nitride capable of attenuating radiation energy, electron beams (β rays) can be prevented from scattering in the support 81 of the stimulable phosphor sheet 80. Accordingly, since the radioactive labeling substance contained in each of a number of the spot-like regions 3 formed in the absorptive substrate 2 of the biochemical analysis unit 1 can selectively exposed to only the corresponding stimulable phosphor layer region 82 of the stimulable phosphor sheet 80, it is possible to produce biochemical analysis data having excellent quantitative characteristics with high resolution by scanning a number of the thus exposed stimulable phosphor layer regions 82 with the laser beam 24 and photoelectrically detecting stimulated emission 45 released from a number of the stimulable phosphor layer regions 82.

On the other hand, since it is possible to effectively prevent electron beams (β rays) released from the radioactive labeling substance contained in the individual spot-like regions 3 formed in the absorptive substrate 2 of the biochemical analysis unit 1 from impinging onto the stripe-shaped additional stimulable phosphor layer regions 85 formed in the support 81 of the stimulable phosphor sheet 80 and the stripe-shaped additional stimulable phosphor layer regions 85 from being exposed, only electron beams (β rays) released from radioactive labeling substance adhering to the surface of the substrate 2 of the biochemical analysis unit 1 during hybridization and remaining even after washing, ambient radiation and the like impinge onto the stripe-shaped additional stimulable phosphor layer regions 85 formed in the support 81 of the stimulable phosphor sheet 80 and the stripe-shaped additional stimulable phosphor layer regions 85 are exposed to only electron beams (β rays) released from radioactive labeling substance adhering to the surface of the substrate 2 of the biochemical analysis unit 1 during hybridization and remaining even after washing, ambient radiation and the like. Therefore, since digital data produced by scanning the stripe-shaped additional stimulable phosphor layer regions 85 with the laser beam 24 and photoelectrically detecting stimulated emission 45 released from the stripe-shaped additional stimulable phosphor layer regions 85 correspond to background noise, it is possible to produce biochemical analysis data free of background noise with high accuracy similarly to the previous embodiment by producing background noise correction data from digital data obtained by photoelectrically detecting stimulated emission 45 released from the stripe-shaped additional stimulable phosphor layer regions 85 and subtracting them from the background noise correction data from digital data obtained by scanning the whole surface of the stimulable phosphor sheet 80.

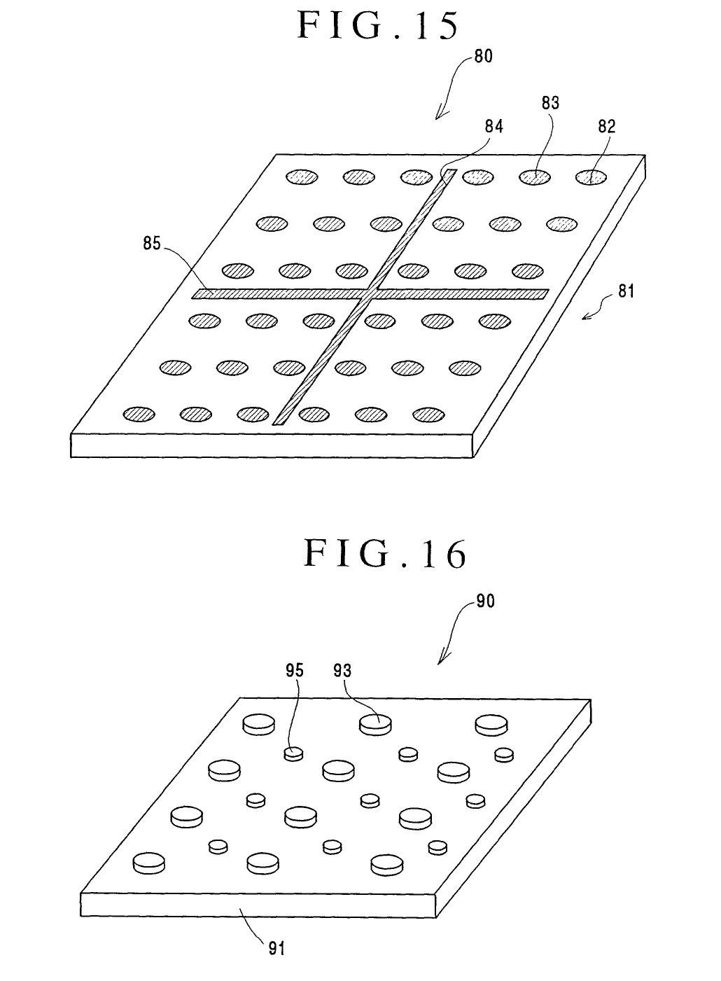

FIG. 16 is a schematic perspective view showing a stimulable phosphor sheet which is a further preferred embodiment of the present invention.

As shown in FIG. 16, a stimulable phosphor sheet 90 according to this embodiment includes a support 91 made of polyethylene terephthalate, a number of stimulable phosphor layer regions 92 formed on the surface of the support 91 and a number of additional stimulable phosphor layer regions 95 regularly formed on the surface of the support 91 between a number of the additional stimulable phosphor layer regions 95.

A number of the stimulable phosphor layer regions 92 are formed on the surface of the support 91 in the same regular pattern as that of a number of the spot-like regions 3 formed in the absorptive substrate 2 of the biochemical analysis unit 1 so that each of them has the same size as that of each of the spot-like regions 3 and a substantially circular shape and the stimulable phosphor sheet 90 is constituted so that each of the stimulable phosphor layer regions 82 faces and abuts against only the corresponding spot-like region 3 formed in the absorptive substrate 2 of the biochemical analysis unit 1.

According to this embodiment, when a number of the stimulable phosphor layer regions 92 formed on the surface of the support 91 of the stimulable phosphor sheet 90 are to be exposed to the radioactive labeling substance selectively contained in a number of the spot-like regions 3 formed in the absorptive substrate 2 of the biochemical analysis unit 1, since the stimulable phosphor sheet 90 is superposed on the biochemical analysis unit 1 in such a manner that each of a number of the stimulable phosphor layer regions 92 formed on the surface of the support 91 of the stimulable phosphor sheet 90 in the same regular pattern as that of a number of the spot-like regions 3 formed in the absorptive substrate 2 of the biochemical analysis unit 1 faces and abuts against the corresponding spot-like region 3 formed in the absorptive substrate 2 of the biochemical analysis unit 1, almost all electron beams (β rays) released from the radioactive labeling substance contained in the individual spot-like regions 3 formed in the absorptive substrate 2 of the biochemical analysis unit 1 impinge only onto the corresponding stimulable phosphor layer regions 92 and, therefore, the radioactive labeling substance selectively contained in the individual spot-like regions 3 formed in the absorptive substrate 2 of the biochemical analysis unit 1 can selectively expose the corresponding stimulable phosphor layer regions 92 of the stimulable phosphor sheet 90. Accordingly, it is possible to produce biochemical analysis data having excellent quantitative characteristics with high resolution by scanning a number of the thus exposed stimulable phosphor layer regions 92 with the laser beam 24 and photoelectrically detecting stimulated emission 45 released from a number of the stimulable phosphor layer regions 92.

On the other hand, since it is possible to effectively prevent electron beams (β rays) released from the radioactive labeling substance contained in the individual spot-like regions 3 formed in the absorptive substrate 2 of the biochemical analysis unit 1 from impinging onto a number of the additional stimulable phosphor layer regions 95 formed on the surface of the support 91 of the stimulable phosphor sheet 90 and the additional stimulable phosphor layer regions 95 from being exposed, only electron beams (β rays) released from radioactive labeling substance adhering to the surface of the substrate 2 of the biochemical analysis unit 1 during hybridization and remaining even after washing, ambient radiation and the like impinge onto a number of the additional stimulable phosphor layer regions 95 formed on the surface of the support 91 of the stimulable phosphor sheet 90 and a number of the additional stimulable phosphor layer regions 95 are exposed to only electron beams (β rays) released from radioactive labeling substance adhering to the surface of the substrate 2 of the biochemical analysis unit 1 during hybridization and remaining even after washing, ambient radiation and the like. Therefore, since digital data produced by scanning a number of the additional stimulable phosphor layer regions 95 with the laser beam 24 and photoelectrically detecting stimulated emission 45 released from a number of the additional stimulable phosphor layer regions 95 correspond to background noise, it is possible to produce biochemical analysis data free of background noise with high accuracy similarly to the previous embodiments by producing background noise correction data from digital data obtained by photoelectrically detecting stimulated emission 45 released from a number of the additional stimulable phosphor layer regions 95 and subtracting them from the background noise correction data from digital data obtained by scanning the whole surface of the stimulable phosphor sheet 90.

The present invention has thus been shown and described with reference to specific embodiments. However, it should be noted that the present invention is in no way limited to the details of the described arrangements but changes and modifications may be made without departing from the scope of the appended claims.

For example, in the above described embodiments, as specific binding substances, cDNAs each of which has a known base sequence and is different from the others are used. However, specific binding substances usable in the present invention are not limited to cDNAs but all specific binding substances capable of specifically binding with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, can be employed in the present invention as a specific binding substance.

Further, in the above described embodiments, specific binding substances are hybridized with substances derived from a living organism labeled with a radioactive labeling substance. However, it is not absolutely necessary to hybridize substances derived from a living organism with specific binding substances and substances derived from a living organism may be specifically bound with specific binding substances by means of antigen-antibody reaction, receptor-ligand reaction or the like instead of hybridization.

Furthermore, in the above described embodiments, although the biochemical analysis unit 1 includes a number of the spot-like regions 3 formed by spotting a solution containing specific binding substances such as a plurality of cDNAs onto the absorptive substrate 2 and selectively hybridizing a substance derived from a living organism labeled with a radioactive labeling substance with the specific binding substances, it is possible to form a biochemical analysis unit 1 by forming a number of through-holes or recesses in a substrate, charging absorptive material such as nylon-6 in a number of the through-holes or recesses to form a number of absorptive regions spaced apart from each other, spotting a solution containing specific binding substances such as a plurality of cDNAs onto a number of the absorptive regions and selectively hybridizing a substance derived from a living organism labeled with a radioactive labeling substance with the specific binding substances contained in a number of the absorptive regions.

Moreover, the support 11 of the stimulable phosphor sheet 10 is made of stainless steel in the embodiment shown in FIGS. 1 to 14, the support 81 of the stimulable phosphor 80 is made of silicon nitride in the embodiment shown in FIG. 15 and the support 91 of the stimulable phosphor sheet 90 is made of polyethylene terephthalate in the embodiment shown in FIG. 16. However, it is not absolutely necessary to form the support 11, 81, 91 of the stimulable phosphor sheet 10, 80, 90 of stainless steel, silicon nitride or polyethylene terephthalate and the support 11, 81, 91 of the stimulable phosphor sheet 10, 80, 90 can be made of other material. The support 11, 81, 91 of the stimulable phosphor sheet 10, 80, 90 is preferably made of material capable of attenuating radiation energy but the material for forming the support 11, 81, 91 of the stimulable phosphor sheet 10, 80, 90 is not particularly limited. The support 11, 81, 91 of the stimulable phosphor sheet 10, 80, 90 can be formed of either inorganic compound material or organic compound material and is preferably formed of metal material, ceramic material or plastic material. Illustrative examples of inorganic compound materials include metals such as gold, silver, copper, zinc, aluminum, titanium, tantalum, chromium, steel, nickel, cobalt, lead, tin, selenium and the like; alloys such as brass, stainless, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. High molecular compounds are preferably used as organic compound material and illustrative examples thereof include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate, polyethylene terephthalate and the like; nylons such as nylon-6, nylon-6,6, nylon-4,10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials.

Further, in the above described embodiments, although the stimulable phosphor layer regions 12, 82, 92 of the stimulable phosphor sheet 10, 80, 90 are formed to have the same size as that of each of the spot-like regions 3 formed in the absorptive substrate 2 of the biochemical analysis unit 1 and to have a substantially circular shape, it is not absolutely necessary to form the stimulable phosphor layer regions 12, 82, 92 of the stimulable phosphor sheet 10, 80, 90 to be substantially circular and the stimulable phosphor layer regions 12, 82, 92 of the stimulable phosphor sheet 10, 80, 90 can be formed to have some other shape such as a substantially rectangular shape. Furthermore, it is not absolutely necessary to form the stimulable phosphor layer regions 12, 82, 92 of the stimulable phosphor sheet 10, 80, 90 to have the same size of that of each of the spot-like regions 3 of the biochemical analysis unit 1.

Moreover, in the above described embodiments, 19,200 of substantially circular spot-like regions 3 having a size of about 0.07 cm² are regularly formed in the absorptive substrate 2 of the biochemical analysis unit 1 and correspondingly, 19,200 of substantially circular stimulable phosphor layer regions 12, 82, 92 having a size of about 0.07 cm² are regularly formed in the support 11, 81, 91 of the stimulable phosphor sheet 10, 80, 90. However, the number or size of the spot-like regions 3 may be arbitrarily selected in accordance with the purpose and correspondingly, the number or size of the stimulable phosphor layer regions 12, 82, 92 may be arbitrarily selected. Preferably, 10 or more of the spot-like regions 3 having a size of 5 cm² or less are formed in the absorptive substrate 2 of the biochemical analysis unit 1 at a density of 10/cm² or less and correspondingly, 10 or more of the stimulable phosphor layer regions 12, 82, 92 having a size of 5 cm² or less are formed in the support 11, 81, 91 of the stimulable phosphor sheet 10, 80, 90.

Further, in the above described embodiments, although the stimulable phosphor layer regions 12, 82, 92 of the stimulable phosphor sheet 10, 80, 90 are formed in the support 11, 81, 91 in the same regular pattern as that of a number of the spot-like regions 3 formed in the absorptive substrate 2 of the biochemical analysis unit 1, it is sufficient for the stimulable phosphor layer regions 12, 82, 92 of the stimulable phosphor sheet 10, 80, 90 to be formed in the same pattern as that of a number of the spot-like regions 3 formed in the absorptive substrate 2 of the biochemical analysis unit 1 and it is not absolutely necessary to form the stimulable phosphor layer regions 12, 82, 92 of the stimulable phosphor sheet 10, 80, 90 in a regular pattern.

Furthermore, in the embodiment shown in FIGS. 1 to 14 and the embodiment shown in FIG. 16, a number of the additional stimulable phosphor layer regions 15, 95 are formed in the support 11, 91 between a number of the stimulable phosphor layer regions 12, 92. However, it is not absolutely necessary to form a number of the additional stimulable phosphor layer regions 15, 95 in the support 11, 91 between a number of the stimulable phosphor layer regions 12, 92 and the arbitrary number of the additional stimulable phosphor layer regions 15, 95 may be formed at arbitrary positions of the support 11, 91 in accordance with the purpose.

Moreover, in the embodiment shown in FIG. 15, although the two stripe-shaped additional stimulable phosphor layer regions 85 are formed so as to be perpendicular to each other by embedding stimulable phosphor in the two grooves 84 formed perpendicularly to each other in the support 81 between a number of the stimulable phosphor layer regions 82, it is not absolutely necessary to form the stripe-shaped additional stimulable phosphor layer regions 85 so as to be perpendicular to each other and the number of the stripe-shaped additional stimulable phosphor layer regions 85 may be arbitrarily selected in accordance with the purpose.

Further, in the embodiment shown in FIGS. 1 to 14 and the embodiment shown in FIG. 16, although a number of the additional stimulable phosphor layer regions 15, 95 are formed in a number of the recesses 14 formed in the support 11 or on the surface of the support 91, instead of the additional stimulable phosphor layer regions 15, 95, similarly to the embodiment shown in FIG. 15, the stripe shaped additional stimulable phosphor layer regions may be formed in the recesses 14 formed in the support 11 or on the surface of the support 91. Furthermore, in the embodiment shown in FIG. 15, although the two stripe-shaped additional stimulable phosphor layer regions 85 are formed so as to be perpendicular to each other in the two grooves 84 formed in the support 81, instead of the two stripe-shaped additional stimulable phosphor layer regions 85 perpendicular to each other, similarly to the embodiment shown in FIGS. 1 to 14 and the embodiment shown in FIG. 16, a number of the additional stimulable phosphor layer regions 15, 95 may be formed in a number of recesses formed in the support 81 or on the surface of the support 81.

Moreover, in the embodiment shown in FIGS. 1 to 14 and the embodiment shown in FIG. 16, although a number of the additional stimulable phosphor layer regions 15, 95 are formed so that the size thereof is smaller than that of the stimulable phosphor layer regions 12, 92, it is not absolutely necessary to form a number of the additional stimulable phosphor layer regions 15, 95 so that the size thereof is smaller than that of the stimulable phosphor layer regions 12, 92 and the size of the additional stimulable phosphor layer regions 15, 95 may be arbitrarily selected in accordance with the purpose. Further, in the embodiment shown in FIGS. 1 to 14, although a number of the stimulable phosphor layer regions 12 are formed by embedding stimulable phosphor in a number of the recesses 13 so that the surfaces of the stimulable phosphor layer regions 12 lie at the same height level as that of the surface of the support 11, it is not absolutely necessary to form a number of the stimulable phosphor layer regions 12 so that the surfaces of the stimulable phosphor layer regions 12 lie at the same height level as that of the surface of the support 11 and the surfaces of the stimulable phosphor layer regions 12 may be positioned below the surface of the support 11 or above the surface of the support 11.

Furthermore, in the embodiment shown in FIG. 15, although the stripe-shaped additional stimulable phosphor layer regions 85 are formed by embedding stimulable phosphor in the grooves 84 formed in the support 81, the stripe-shaped additional stimulable phosphor layer regions 85 may be formed by embedding stimulable phosphor in slots formed in the support 81.

Moreover, in the embodiment shown in FIGS. 1 to 14, although a number of the additional stimulable phosphor layer regions 12 are formed by embedding stimulable phosphor in a number of the recesses 14 formed in the support 11, a number of the additional stimulable phosphor layer regions 12 may be formed by forming a number of through-holes in the support 11 instead of the recesses 14 and embedding stimulable phosphor in a number of the through-holes.

According to the present invention, it is possible to provide a stimulable phosphor sheet and a method for reading biochemical analysis data recorded in a stimulable phosphor sheet which can produce biochemical analysis data having excellent quantitative characteristics with high resolution even in the case of forming at a high density on the surface of a carrier a plurality of spot-like regions containing specific binding substances which can specifically bind with a substance derived from a living organism and whose sequence, base length, composition and the like are known, and specifically binding a substance derived from a living organism labeled with a radioactive labeling substance with specific binding substances contained in the plurality of spot-like regions, thereby selectively labeling the plurality of spot-like regions.

1. A stimulable phosphor sheet including a support formed with a plurality of stimulable phosphor layer regions spaced apart from each other and at least one additional stimulable phosphor layer region spaced apart from the plurality of stimulable phosphor layer regions.

2. A stimulable phosphor sheet in accordance with claim 1 wherein the support of the stimulable phosphor sheet is formed with a plurality of holes spaced apart from each other and the plurality of stimulable phosphor layer regions are formed by charging stimulable phosphor in the plurality of holes.

3. A stimulable phosphor sheet in accordance with claim 2 wherein the support of the stimulable phosphor sheet is formed with a plurality of through-holes spaced apart from each other and the plurality of stimulable phosphor layer regions are formed by charging stimulable phosphor in the plurality of through-holes.

4. A stimulable phosphor sheet in accordance with claim 2 wherein the support of the stimulable phosphor sheet is formed with a plurality of recesses spaced apart from each other and the plurality of stimulable phosphor layer regions are formed by charging stimulable phosphor in the plurality of recesses.

5. A stimulable phosphor sheet in accordance with claim 1 wherein the plurality of stimulable phosphor layer regions of the stimulable phosphor sheet are formed on the surface of the support of the stimulable phosphor sheet.

6. A stimulable phosphor sheet in accordance with of claim 1 wherein the plurality of stimulable phosphor layer regions of the stimulable phosphor sheet are dot-like formed in the support.

7. A stimulable phosphor sheet in accordance with of claim 2 wherein the plurality of stimulable phosphor layer regions of the stimulable phosphor sheet are dot-like formed in the support.

8. A stimulable phosphor sheet in accordance with claim 1 wherein a plurality of the additional stimulable phosphor layer regions are dot-like formed in the support of the stimulable phosphor sheet.

9. A stimulable phosphor sheet in accordance with claim 2 wherein a plurality of the additional stimulable phosphor layer regions are dot-like formed in the support of the stimulable phosphor sheet.

10. A stimulable phosphor sheet in accordance with claim 8 wherein the plurality of the additional stimulable phosphor layer regions are dot-like formed in the support of the stimulable phosphor sheet between at least some of the plurality of stimulable phosphor layer regions.

11. A stimulable phosphor sheet in accordance with claim 9 wherein the plurality of the additional stimulable phosphor layer regions are dot-like formed in the support of the stimulable phosphor sheet between at least some of the plurality of stimulable phosphor layer regions.

12. A stimulable phosphor sheet in accordance with claim 1 wherein the at least one additional stimulable phosphor layer region of the stimulable phosphor sheet is formed in a stripe shape in the support.

13. A stimulable phosphor sheet in accordance with claim 2 wherein the at least one additional stimulable phosphor layer region of the stimulable phosphor sheet is formed in a stripe shape in the support.

14. A stimulable phosphor sheet in accordance with claim 12 wherein the at least one additional stimulable phosphor layer regions of the stimulable phosphor sheet is formed in a stripe shape in the support between at least some of the plurality of stimulable phosphor layer regions.

15. A stimulable phosphor sheet in accordance with claim 13 wherein the at least one additional stimulable phosphor layer regions of the stimulable phosphor sheet is formed in a stripe shape in the support between at least some of the plurality of stimulable phosphor layer regions.

16. A stimulable phosphor sheet in accordance with claim 1 wherein each of the additional stimulable phosphor layer regions of the stimulable phosphor sheet is formed so as to have a smaller size than that of each of the plurality of stimulable phosphor layer regions.

17. A stimulable phosphor sheet in accordance with claim 2 wherein each of the additional stimulable phosphor layer regions of the stimulable phosphor sheet is formed so as to have a smaller size than that of each of the plurality of stimulable phosphor layer regions.

18. A stimulable phosphor sheet in accordance with claim 1 wherein the support of the stimulable phosphor sheet is formed of a material capable of attenuating radiation energy.

19. A stimulable phosphor sheet in accordance with claim 2 wherein the support of the stimulable phosphor sheet is formed of a material capable of attenuating radiation energy.

20. A stimulable phosphor sheet in accordance with claim 18 wherein the support of the stimulable phosphor sheet is made of a material of reducing the energy of radiation to ⅕ or less when the radiation travels in the support by a distance equal to that between neighboring stimulable phosphor layer regions.

21. A stimulable phosphor sheet in accordance with claim 19 wherein the support of the stimulable phosphor sheet is made of a material of reducing the energy of radiation to ⅕ or less when the radiation travels in the support by a distance equal to that between neighboring stimulable phosphor layer regions.

22. A stimulable phosphor sheet in accordance with claim 20 wherein the support of the stimulable phosphor sheet is made of a material of reducing the energy of radiation to 1/10 or less when the radiation travels in the support by a distance equal to that between neighboring stimulable phosphor layer regions.

23. A stimulable phosphor sheet in accordance with claim 21 wherein the support of the stimulable phosphor sheet is made of a material of reducing the energy of radiation to 1/10 or less when the radiation travels in the support by a distance equal to that between neighboring stimulable phosphor layer regions.

24. A stimulable phosphor sheet in accordance with claim 22 wherein the support of the stimulable phosphor sheet is made of a material of reducing the energy of radiation to 1/100 or less when the radiation travels in the support by a distance equal to that between neighboring stimulable phosphor layer regions.

25. A stimulable phosphor sheet in accordance with claim 23 wherein the support of the stimulable phosphor sheet is made of a material of reducing the energy of radiation to 1/100 or less when the radiation travels in the support by a distance equal to that between neighboring stimulable phosphor layer regions.

26. A stimulable phosphor sheet in accordance with claim 20 wherein the support is made of a material selected from a group consisting a metal material, a ceramic material and a plastic material.

27. A stimulable phosphor sheet in accordance with claim 21 wherein the support is made of a material selected from a group consisting a metal material, a ceramic material and a plastic material.

28. A method for reading biochemical analysis data recorded in a stimulable phosphor sheet comprising the steps of superposing a stimulable phosphor sheet including a support formed with a plurality of stimulable phosphor layer regions spaced apart from each other and at least one additional stimulable phosphor layer region spaced apart from the plurality of stimulable phosphor layer regions and a biochemical analysis unit including a plurality of spot-like regions formed by spotting specific binding substances whose sequence, base length, composition and the like are known and specifically binding a substance derived from a living organism labeled with a radioactive labeling substance with the specific binding substances, thereby selectively labeling the the plurality of spot-like regions with the radioactive labeling substance, exposing the plurality of stimulable phosphor layer regions of the stimulable phosphor sheet to the radioactive labeling substance selectively contained in the plurality of spot-like regions, irradiating the plurality of stimulable phosphor layer regions and the at least one additional stimulable phosphor layer region of the stimulable phosphor sheet with a stimulating ray, thereby exciting stimulable phosphor contained in the plurality of stimulable phosphor layer regions and the at least one additional stimulable phosphor layer region, photoelectrically detecting stimulated emission released from the stimulable phosphor to produce analog data, digitizing the analog data to produce digital data and subtracting digital data obtained by irradiating the at least one additional stimulable phosphor layer region with the stimulating ray and photoelectrically detecting stimulated emission released therefrom from digital data obtained by irradiating the plurality of stimulable phosphor layer regions with the stimulating ray and photoelectrically detecting stimulated emission released therefrom, thereby producing biochemical analysis data.

29. A method for reading biochemical analysis data recorded in a stimulable phosphor sheet in accordance with claim 28 wherein the support of the stimulable phosphor sheet is formed with a plurality of holes spaced apart from each other and the plurality of stimulable phosphor layer regions are formed by charging stimulable phosphor in the plurality of holes.

30. A method for reading biochemical analysis data recorded in a stimulable phosphor sheet in accordance with claim 28 wherein the plurality of stimulable phosphor layer regions of the stimulable phosphor sheet are formed on the surface of the support of the stimulable phosphor sheet.

31. A method for reading biochemical analysis data recorded in a stimulable phosphor sheet in accordance with claim 28 wherein the plurality of stimulable phosphor layer regions of the stimulable phosphor sheet are dot-like formed in the support.

32. A method for reading biochemical analysis data recorded in a stimulable phosphor sheet in accordance with claim 29 wherein the plurality of stimulable phosphor layer regions of the stimulable phosphor sheet are dot-like formed in the support.

33. A method for reading biochemical analysis data recorded in a stimulable phosphor sheet in accordance with claim 28 wherein a plurality of the additional stimulable phosphor layer regions are dot-like formed in the support of the stimulable phosphor sheet.

34. A method for reading biochemical analysis data recorded in a stimulable phosphor sheet in accordance with claim 29 wherein a plurality of the additional stimulable phosphor layer regions are dot-like formed in the support of the stimulable phosphor sheet.

35. A method for reading biochemical analysis data recorded in a stimulable phosphor sheet in accordance with claim 33 wherein the plurality of the additional stimulable phosphor layer regions are dot-like formed in the support of the stimulable phosphor sheet between at least some of the plurality of stimulable phosphor layer regions.

36. A method for reading biochemical analysis data recorded in a stimulable phosphor sheet in accordance with claim 34 wherein the plurality of the additional stimulable phosphor layer regions are dot-like formed in the support of the stimulable phosphor sheet between at least some of the plurality of stimulable phosphor layer regions.

37. A method for reading biochemical analysis data recorded in a stimulable phosphor sheet in accordance with claim 28 wherein the at least one additional stimulable phosphor layer region of the stimulable phosphor sheet is formed in a stripe shape in the support.

38. A method for reading biochemical analysis data recorded in a stimulable phosphor sheet in accordance with claim 29 wherein the at least one additional stimulable phosphor layer region of the stimulable phosphor sheet is formed in a stripe shape in the support.

39. A method for reading biochemical analysis data recorded in a stimulable phosphor sheet in accordance with claim 37 wherein the at least one additional stimulable phosphor layer regions of the stimulable phosphor sheet is formed in a stripe shape in the support between at least some of the plurality of stimulable phosphor layer regions.

40. A method for reading biochemical analysis data recorded in a stimulable phosphor sheet in accordance with claim 38 wherein the at least one additional stimulable phosphor layer regions of the stimulable phosphor sheet is formed in a stripe shape in the support between at least some of the plurality of stimulable phosphor layer regions.

41. A method for reading biochemical analysis data recorded in a stimulable phosphor sheet in accordance with claim 28 wherein each of the additional stimulable phosphor layer regions of the stimulable phosphor sheet is formed so as to have a smaller size than that of each of the plurality of stimulable phosphor layer regions.

42. A method for reading biochemical analysis data recorded in a stimulable phosphor sheet in accordance with claim 29 wherein each of the additional stimulable phosphor layer regions of the stimulable phosphor sheet is formed so as to have a smaller size than that of each of the plurality of stimulable phosphor layer regions.

43. A method for reading biochemical analysis data recorded in a stimulable phosphor sheet in accordance with claim 28 wherein the support of the stimulable phosphor sheet is formed of a material capable of attenuating radiation energy.

44. A method for reading biochemical analysis data recorded in a stimulable phosphor sheet in accordance with claim 29 wherein the support of the stimulable phosphor sheet is formed of a material capable of attenuating radiation energy.

45. A method for reading biochemical analysis data recorded in a stimulable phosphor sheet in accordance with claim 43 wherein the support of the stimulable phosphor sheet is made of a material of reducing the energy of radiation to ⅕ or less when the radiation travels in the support by a distance equal to that between neighboring stimulable phosphor layer regions.

46. A method for reading biochemical analysis data recorded in a stimulable phosphor sheet in accordance with claim 44 wherein the support of the stimulable phosphor sheet is made of a material of reducing the energy of radiation to ⅕ or less when the radiation travels in the support by a distance equal to that between neighboring stimulable phosphor layer regions.

47. A method for reading biochemical analysis data recorded in a stimulable phosphor sheet in accordance with claim 45 wherein the support is made of a material selected from a group consisting a metal material, a ceramic material and a plastic material.

48. A method for reading biochemical analysis data recorded in a stimulable phosphor sheet in accordance with claim 46 wherein the support is made of a material selected from a group consisting a metal material, a ceramic material and a plastic material.
